(19) United States
(12) Patent Application Publication
(10) Pub. No.: US 2025/0263659 A1
(43) Pub. Date: Aug. 21, 2025

(54) METHOD AND KIT FOR PREPARING AN IMMUNE CELL DIFFERENTIATED FROM A STEM CELL

(71) Applicant: INDUSTRIAL TECHNOLOGY RESEARCH INSTITUTE, Hsinchu (TW)

(72) Inventors: Mei-Wei LIN, Zhubei City (TW); Yen-Ju LIN, Zhudong Township (TW); Min-Yuan CHOU, Taipei City (TW); Chung-Yuan SUN, Zhudong Township (TW); Li-Tsen LIN, New Taipei City (TW)

(73) Assignee: INDUSTRIAL TECHNOLOGY RESEARCH INSTITUTE, Hsinchu (TW)

(21) Appl. No.: 19/003,202

(22) Filed: Dec. 27, 2024

Related U.S. Application Data
(60) Provisional application No. 63/615,465, filed on Dec. 28, 2023.

(51) Int. Cl. C12N 5/0783

(52) U.S. Cl. CPC C12N 5/0636; C12N 2501/125; C12N 2501/145; C12N 2501/2303; C12N 2501/2306; C12N 2501/2307; C12N 2501/26; C12N 2501/42; C12N 2501/58; C12N 2506/11

(57) ABSTRACT

A method for preparing an immune cell differentiated from a stem cell is provided. The method includes: (a) forming a coating containing a matrix on a surface; and (b) culturing a stem cell on the coating in the presence of a first cytokine combination to differentiate the stem cell into an immune cell, wherein the immune cell includes a γδ T cell (gamma delta T cell, GDT cell). The matrix includes: vascular cell adhesion molecule (VCAM); intercellular adhesion molecule (ICAM); and delta-like ligand 4 (DLL4). The first cytokine combination includes: stem cell factor (SCF); thrombopoietin (TPO); Fms-like tyrosine kinase 3 ligand (Flt3L); and interleukin-7 (IL-7). Moreover, a source of the stem cell includes umbilical cord blood or an induced pluripotent stem cell (iPSC), and the stem cell is CD34 positive.

### CROSS REFERENCE TO RELATED APPLICATIONS

This application claims the benefit of U.S. Provisional Application No. 63/615,465, filed on Dec. 28, 2023, the entirety of which is incorporated by reference herein.

### TECHNICAL FIELD

The present invention relates to the differentiation of stem cells, and in particular it relates to methods and kits for preparing immune cells that are differentiated from stem cells.

### BACKGROUND

Cellular immunotherapy is a new trend in current cancer treatment. However, the culture and expansion technology using autologous immune cells often results in unsatisfactory treatment results due to the poor functioning of the patient's immune cells, and requires more costly quality and process control, making it unaffordable for most patients. Therefore, the development of high-quality immune cells that can be used allogeneically (allogenic therapy) will be an important direction for improving the therapeutic efficacy of immune cells.

Hematopoietic stem cells (HPSCs) are a population of stem cells in the blood that have the potential to differentiate into other blood cells. Hematopoietic stem cells are present in only a small amount in adult whole blood, however, they are present in higher amounts in umbilical cord blood. According to overseas research surveys, it is found that the utilization rate of umbilical cord blood is less than 1 in 10,000 per person. Therefore, if an in vitro and efficient methodology can be developed to differentiate hematopoietic stem cells into therapeutic immune cells, such as γδ T cells (gamma delta T cells, GDT cells), NK cells, it will expand the scope of applications for preserved umbilical cord blood and spur the development of the umbilical cord blood industry.

Current studies have shown that whether it is in vivo proliferation, in vitro stimulated proliferation and transfusion, or autologous or allogeneic transfusion, serious clinical adverse events are usually not directly related to γδ T-cell therapy, and thus γδ T-cell-based cellular immunotherapy is relatively safe in clinical practice. In addition, cellular immunotherapy with γδ T cells does not usually require a lymphocyte depletion step. Therefore, cellular immunotherapy of γδ T cells is one of the key development targets of current cell therapy.

In the current existing technology, γδ T cells are mostly obtained using a peripheral blood separation method, however, the initial number of γδ T cells that can be obtained using this method is small, and a sufficient therapeutic dose must be obtained through an in vitro expansion method.

Accordingly, how to obtain a sufficient therapeutic dosage of γδ T cells in a simple way is an urgent issue and a research direction that needs to be solved at present.

### SUMMARY

The present disclosure provides a method for preparing an immune cell differentiated from a stem cell, comprising: (a) forming a coating containing a matrix on a surface; and (b) culturing a stem cell on the coating in the presence of a first cytokine combination to differentiate the stem cell into an immune cell, wherein the immune cell includes a γδ T cell (gamma delta T cell, GDT cell). The matrix comprises: vascular cell adhesion molecule (VCAM); intercellular adhesion molecule (ICAM); and delta-like ligand 4 (DLL4). The first cytokine combination comprises: stem cell factor (SCF); thrombopoietin (TPO); Fms-like tyrosine kinase 3 ligand (Flt3L); and interleukin-7 (IL-7). Moreover, a source of the stem cell includes umbilical cord blood or an induced pluripotent stem cell (iPSC), and the stem cell is CD34 positive.

In addition, the present disclosure also provides a kit for preparing an immune cell differentiated from a stem cell, comprising: a coating formation sub-kit for forming a coating on a surface; and a stem cell differentiation sub-kit. The coating formation sub-kit comprises: a matrix component for forming a coating formation solution with a solvent. The matrix component comprises: vascular cell adhesion molecule; intercellular adhesion molecule; and delta-like ligand 4, and the coating formation solution is used to coat the surface to form the coating on the surface. Moreover, the stem cell differentiation sub-kit comprises: a first cytokine combination for adding to a medium to form a stem cell differentiation medium. The first cytokine combination comprises: stem cell factor; thrombopoietin; Fms-like tyrosine kinase 3 ligand; and interleukin-7.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows CD34 expression of hematopoietic stem cells (HSCs) isolated from umbilical cord blood of the donor;

FIG. 2A shows expansion folds of hematopoietic stem cells isolated from umbilical cord blood of the donor with code HUCB0100919M and the donor with code HUCB012023A after 6 days of culture in the presence of human stem cell factor (SCF), human Fms-like tyrosine kinase 3 ligand (Flt3L), human interleukin-3 (IL-3) and human interleukin-6 (IL-6);

FIG. 2B shows CD45 expression and CD34 expression of hematopoietic stem cells isolated from umbilical cord blood of the donor with code HUCB0100919M after 6 days of culture in the presence of human stem cell factor (SCF), human Fms-like tyrosine kinase 3 ligand (Flt3L), human interleukin-3 (IL-3) and human interleukin-6 (IL-6);

FIG. 2C shows CD45 expression and CD34 expression of hematopoietic stem cells isolated from umbilical cord blood of the donor with code HUCB012023A after 6 days of culture in the presence of human stem cell factor (SCF), human Fms-like tyrosine kinase 3 ligand (Flt3L), human interleukin-3 (IL-3) and human interleukin-6 (IL-6);

FIG. 3 shows expression of γδ TCR of expanded hematopoietic stem cells after 14 days of culture with a culture plate with a coating containing vascular cell adhesion molecule (VCAM), intercellular adhesion molecule (ICAM) and delta-like ligand 4 (DLL4) in the presence of stem cell factor (SCF), thrombopoietin (TPO), Fms-like tyrosine kinase 3 ligand (Flt3L) and interleukin-7 (IL-7);

FIG. 4A shows evaluation results of the toxic effect of γδ T cells differentiated from hematopoietic stem cells on lymphoma cancer cells K562 by Calcein AM staining;

FIG. 4B shows evaluation results of the toxic effect of γδ T cells differentiated from hematopoietic stem cells on breast cancer cells MDA-MB-231 by Calcein AM staining;

FIG. 5A shows toxic effect of γδ T cells differentiated from hematopoietic stem cells subjected to 13 days of differentiation culture on breast cancer cells MDA-MB-231 analyzed by a real-time cell analyzer (RTCA);

FIG. 5B shows toxic effect of γδ T cells differentiated from hematopoietic stem cells subjected to 26 days of differentiation culture on breast cancer cells MDA-MB-231 analyzed by real-time cell analyzer;

FIG. 6 shows expressions of CD7, CD34, CD56 and γδ TCR of cells differentiated from hematopoietic stem cells subjected to different differentiation culture conditions (different culture medium and with or without intercellular adhesion molecule (ICAM) in the coating); and

FIG. 7 shows expressions of CD34, CD56 and γδ TCR of cells differentiated from hematopoietic stem cells subjected to different differentiation culture conditions (with or without delta-like ligand 4 (DLL4) in the coating).

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure may provide a method for preparing an immune cell differentiated from a stem cell.

The method for preparing an immune cell differentiated from a stem cell provided by the present disclosure may effectively differentiate a stem cell into an immune cell, especially a T cell.

The method for preparing an immune cell differentiated from a stem cell of the present disclosure mentioned above may comprise the following step, but it is not limited thereto.

First, a coating containing a matrix is formed on a surface. The surface mentioned above may comprise, but is not limited to, at least one surface of a cell culture device. Examples of the cell culture device mentioned above may include, but are not limited to, a cell culture container, a cell culture membrane, and a cell culture scaffold, but they are not limited thereto.

In one embodiment, the cell culture device mentioned above may be a cell culture container, and the at least one surface mentioned above may include an inner surface of the cell culture container. In this embodiment, the inner surface mentioned above may include a bottom surface and/or a side wall surface in the cell culture container, but it is not limited thereto. In another embodiment, the cell culture device mentioned above may be a cell culture membrane, and the at least one surface mentioned above may include an upper surface and/or a lower surface of the cell culture membrane. In yet another embodiment, the cell culture device mentioned above may be a cell culture scaffold, and the at least one surface mentioned above may include an outer surface of the cell culture scaffold and/or a surface within a hole of the cell culture scaffold.

The matrix mentioned above may comprise vascular cell adhesion molecule (VCAM), intercellular adhesion molecule (ICAM) and delta-like ligand 4 (DLL4), but it is not limited thereto.

Furthermore, a manner for forming a coating on the aforementioned surface is not particularly limited, as long as a coating containing the aforementioned matrix can be formed on the aforementioned surface. In one embodiment, the coating mentioned above may be formed by a coating formation solution, and the coating formation solution mentioned above may contain the matrix mentioned above. A manner for forming the coating by the coating formation solution also has no particular limitation, as long as the coating formation solution can form a coating containing the aforementioned matrix on the aforementioned surface, ultimately. In one embodiment, the coating formation solution mentioned above is brought into contact with the surface mentioned above, and the coating formation solution mentioned above is covered on the surface mentioned above, so as to form a coating containing the matrix mentioned above on the surface mentioned above.

In one embodiment, in the foregoing coating formation solution, the concentration of vascular cell adhesion molecule in the matrix may be about 0.05-50 μg/mL, such as about 0.1-50 μg/mL, about 0.5-45 μg/mL, about 0.5-40 μg/mL, about 0.75-35 μg/mL, about 1-30 μg/mL, about 2-25 μg/mL, about 2.5-20 μg/mL, about 4-15 μg/mL, about 5-10 μg/mL, about 0.1-10 μg/mL, about 0.05 μg/mL, about 0.06 μg/mL, about 0.07 μg/mL, about 0.08 μg/mL, about 0.09 μg/mL, about 0.1 μg/mL, about 0.2 μg/mL, about 0.25 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1 μg/mL, about 2 μg/mL, about 2.5 μg/mL, about 3 μg/mL, about 4 μg/mL, about 5 μg/mL, about 6 μg/mL, about 7 μg/mL, about 8 μg/mL, about 9 μg/mL, about 10 μg/mL, about 12.5 μg/mL, about 15 μg/mL, about 20 μg/mL, about 25 μg/mL, about 30 μg/mL, about 35 μg/mL, about 40 μg/mL, about 45 μg/mL, about 50 μg/mL, but it is not limited thereto. In one specific embodiment, in the foregoing coating formation solution, the concentration of vascular cell adhesion molecule in the matrix may be about 10 μg/mL. In another specific embodiment, in the foregoing coating formation solution, the concentration of vascular cell adhesion molecule in the matrix may be about 0.1 μg/mL. In yet another specific embodiment, in the foregoing coating formation solution, the concentration of vascular cell adhesion molecule in the matrix may be about 1 μg/mL.

Moreover, in one embodiment, in the foregoing coating formation solution, the concentration of intercellular adhesion molecule in the matrix may be about 0.05-50 μg/mL, such as about 0.1-50 μg/mL, about 0.5-45 μg/mL, about 0.5-40 μg/mL, about 0.75-35 μg/mL, about 1-30 μg/mL, about 2-25 μg/mL, about 2.5-20 μg/mL, about 4-15 μg/mL, about 5-10 μg/mL, about 0.1-10 μg/mL, about 0.05 μg/mL, about 0.06 μg/mL, about 0.07 μg/mL, about 0.08 μg/mL, about 0.09 μg/mL, about 0.1 μg/mL, about 0.2 μg/mL, about 0.25 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1 μg/mL, about 2 μg/mL, about 2.5 μg/mL, about 3 μg/mL, about 4 μg/mL, about 5 μg/mL, about 6 μg/mL, about 7 μg/mL, about 8 μg/mL, about 9 μg/mL, about 10 μg/mL, about 12.5 μg/mL, about 15 μg/mL, about 20 μg/mL, about 25 μg/mL, about 30 μg/mL, about 35 μg/mL, about 40 μg/mL, about 45 μg/mL, about 50 μg/mL, but it is not limited thereto. In one specific embodiment, in the foregoing coating formation solution, the concentration of intercellular adhesion molecule in the matrix may be about 10 μg/mL. In another specific embodiment, in the foregoing coating formation solution, the concentration of intercellular adhesion molecule in the matrix may be about 0.1 μg/mL. In yet another specific embodiment, in the foregoing coating formation solution, the concentration of intercellular adhesion molecule in the matrix may be about 1 μg/mL.

Furthermore, in one embodiment, in the foregoing coating formation solution, the concentration of delta-like ligand 4 in the matrix may be about 0.05-50 μg/mL, such as about 0.1-50 μg/mL, about 0.5-45 μg/mL, about 0.5-40 μg/mL, about 0.75-35 μg/mL, about 1-30 μg/mL, about 2-25 μg/mL, about 2.5-20 μg/mL, about 4-15 μg/mL, about 5-10 μg/mL, about 0.1-10 μg/mL, about 0.05 μg/mL, about 0.06 μg/mL, about 0.07 μg/mL, about 0.08 μg/mL, about 0.09 μg/mL, about 0.1 μg/mL, about 0.2 μg/mL, about 0.25 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1 μg/mL, about 2 μg/mL, about 2.5 μg/mL, about 3 μg/mL, about 4 μg/mL, about 5 μg/mL, about 6 μg/mL, about 7 μg/mL, about 8 μg/mL, about 9 μg/mL, about 10 μg/mL, about 12.5 μg/mL, about 15 μg/mL, about 20 μg/mL, about 25 μg/mL, about 30 μg/mL, about 35 μg/mL, about 40 μg/mL, about 45 μg/mL, about 50 μg/mL, but it is not limited thereto. In one specific embodiment, in the foregoing coating formation solution, the concentration of delta-like ligand 4 in the matrix may be about 10 μg/mL. In another specific embodiment, in the foregoing coating formation solution, the concentration of delta-like ligand 4 in the matrix may be about 0.1 μg/mL. In yet another specific embodiment, in the foregoing coating formation solution, the concentration of delta-like ligand 4 in the matrix may be about 1 μg/mL.

In another embodiment, in the foregoing coating formation solution, the concentration of vascular cell adhesion molecule in the matrix, the concentration of intercellular adhesion molecule in the matrix and the concentration of delta-like ligand 4 in the matrix may independently be about 0.05-50 μg/mL, such as about 0.1-50 μg/mL, about 0.5-45 μg/mL, about 0.5-40 μg/mL, about 0.75-35 μg/mL, about 1-30 μg/mL, about 2-25 μg/mL, about 2.5-20 μg/mL, about 4-15 μg/mL, about 5-10 μg/mL, about 0.1-10 μg/mL, about 0.05 μg/mL, about 0.06 μg/mL, about 0.07 μg/mL, about 0.08 μg/mL, about 0.09 μg/mL, about 0.1 μg/mL, about 0.2 μg/mL, about 0.25 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1 μg/mL, about 2 μg/mL, about 2.5 μg/mL, about 3 μg/mL, about 4 μg/mL, about 5 μg/mL, about 6 μg/mL, about 7 μg/mL, about 8 μg/mL, about 9 μg/mL, about 10 μg/mL, about 12.5 μg/mL, about 15 μg/mL, about 20 μg/mL, about 25 μg/mL, about 30 μg/mL, about 35 μg/mL, about 40 μg/mL, about 45 μg/mL, about 50 μg/mL, but they are not limited thereto. In one specific embodiment, in the foregoing coating formation solution, the concentration of vascular cell adhesion molecule in the matrix, the concentration of intercellular adhesion molecule in the matrix and the concentration of delta-like ligand 4 in the matrix may all be about 10 μg/mL. In another specific embodiment, in the foregoing coating formation solution, the concentration of vascular cell adhesion molecule in the matrix, the concentration of intercellular adhesion molecule in the matrix and the concentration of delta-like ligand 4 in the matrix may all be about 0.1 μg/mL. In yet another specific embodiment, in the foregoing coating formation solution, the concentration of vascular cell adhesion molecule in the matrix, the concentration of intercellular adhesion molecule in the matrix and the concentration of delta-like ligand 4 in the matrix may all be about 1 μg/mL.

In addition, in one embodiment, in the foregoing coating formation solution or the foregoing coating, the content ratio of vascular cell adhesion molecule in the matrix, intercellular adhesion molecule in the matrix and delta-like ligand 4 in the matrix may be about 0.01-50:0.01-50:0.01-50, such as about 0.05-50:0.05-50:0.05-50, about 0.1-45:0.1-45:0.1-45, about 0.5-40:0.5-40:0.5-40, about 1-35:1-35:1-35, about 2-30:2-30:2-30, about 2.5-25:2.5-25:2.5-25, about 4-20:4-20:4-20, about 5-15:5-15:5-15 [symbol] 0.1-10:0.1-10:0.1-10, about 1:1:1, about 1:2:1, about 1:1:2, about 1:2:2, about 2:1:1, about 2:2:1, about 1:3:1, about 1:1:3, about 1:3:3, about 3:1:1, about 3:3:1, about 1:4:1, about 1:1:4, about 1:4:4, about 4:1:1, about 4:4:1, about 1:5:1, about 1:1:5, about 1:5:5, about 5:1:1, about 5:5:1, about 1:6:1, about 1:1:6, about 1:6:6, about 6:1:1, about 6:6:1, about 1:7:1, about 1:1:7, about 1:7:7, about 7:1:1, about 7:7:1, about 1:8:1, about 1:1:8, about 1:8:8, about 8:1:1, about 8:8:1, about 1:9:1, about 1:1:9, about 1:9:9, about 9:1:1, about 9:9:1, about 1:10:1, about 1:1:10, about 1:10:10, about 10:1:1, about 10:10:1, about 1:50:1, about 1:1:50, about 1:50:50, about 50:1:1, about 50:50:1, about 1:100:1, about 1:1:100, about 1:100:100, about 100:1:1, about 100:100:1, but it is not limited thereto. In one specific embodiment, in the foregoing coating formation solution or the foregoing coating, the content ratio of vascular cell adhesion molecule in the matrix, intercellular adhesion molecule in the matrix and delta-like ligand 4 in the matrix may be about 1:1:1.

Furthermore, in the method for preparing an immune cell differentiated from a stem cell of the present disclosure, after the step of forming a coating containing a matrix on a surface, a stem cell is cultured on the coating mentioned above in the presence of a first cytokine combination to differentiate the stem cell mentioned above into an immune cell.

A source of the stem cell mentioned above may comprise umbilical cord blood or an induced pluripotent stem cell (iPSC), but it is not limited thereto. Moreover, the stem cell mentioned above may be CD34 positive, but it is also not limited thereto. In one embodiment, the stem cell mentioned above is a hematopoietic stem cell (HSC). Furthermore, in one embodiment, the immune cell mentioned above may comprise a γδ T cell (gamma delta T cell (GDT cell), a NK cell, etc., but it is not limited thereto.

Moreover, the first cytokine combination mentioned above may comprise, but is not limited to, stem cell factor (SCF), thrombopoietin (TPO), Fms-like tyrosine kinase 3 ligand (Flt3L) and interleukin-7 (IL-7).

In one embodiment, in the foregoing step of culturing a stem cell on the foregoing coating in the presence of a first cytokine combination to differentiate the foregoing stem cell into an immune cell, the concentration of stem cell factor may be about 1-500 ng/mL, such as about 5-450 ng/mL, about 10-400 ng/ml, about 15-350 ng/mL, about 20-300 ng/ml, about 25-250 ng/mL, about 30-200 ng/mL, about 35-150 ng/mL, about 40-100 ng/ml, about 45-95 ng/mL, about 50-90 ng/mL, about 55-85 ng/ml, about 60-80 ng/ml, about 2 ng/ml, about 3 ng/mL, about 4 ng/mL, about 5 ng/mL, about 6 ng/ml, about 7 ng/ml, about 8 ng/ml, about 9 ng/mL, about 10 ng/mL, about 15 ng/mL, about 20 ng/mL, about 25 ng/mL, about 30 ng/mL, about 35 ng/mL, about 40 ng/ml, about 45 ng/ml, about 50 ng/ml, about 55 ng/mL, about 60 ng/mL, about 65 ng/mL, about 70 ng/mL, about 75 ng/mL, about 80 ng/ml, about 85 ng/mL, about 90 ng/mL, about 95 ng/mL, about 100 ng/mL, about 125 ng/ml, about 150 ng/ml, about 200 ng/mL, about 225 ng/mL, about 250 ng/mL, about 300 ng/ml, about 350 ng/mL, about 400 ng/mL, about 450 ng/mL, about 500 ng/mL, but it is not limited thereto. In one specific embodiment, the concentration of stem cell factor may be about 100 ng/mL. In another specific embodiment, the concentration of stem cell factor may be about 8 ng/ml. In yet another embodiment, the concentration of stem cell factor may be about 20 ng/mL. Furthermore, in another embodiment, the concentration of stem cell factor may be about 50 ng/mL.

Furthermore, in one embodiment, in the foregoing step of culturing a stem cell on the foregoing coating in the presence of a first cytokine combination to differentiate the foregoing stem cell into an immune cell, the concentration of thrombopoietin may be about 1-500 ng/mL, such as about 5-450 ng/ml, about 10-400 ng/ml, about 15-350 ng/mL, about 20-300 ng/mL, about 25-250 ng/mL, about 30-200 ng/ml, about 35-150 ng/ml, about 40-100 ng/mL, about 45-95 ng/mL, about 50-90 ng/ml, about 55-85 ng/ml, about 60-80 ng/ml, about 2 ng/mL, about 3 ng/mL, about 4 ng/mL, about 5 ng/mL, about 6 ng/mL, about 7 ng/ml, about 8 ng/mL, about 9 ng/mL, about 10 ng/mL, about 15 ng/ml, about 20 ng/ml, about 25 ng/mL, about 30 ng/mL, about 35 ng/mL, about 40 ng/mL, about 45 ng/ml, about 50 ng/ml, about 55 ng/mL, about 60 ng/mL, about 65 ng/mL, about 70 ng/mL, about 75 ng/ml, about 80 ng/mL, about 85 ng/mL, about 90 ng/ml, about 95 ng/mL, about 100 ng/ml, about 125 ng/mL, about 150 ng/mL, about 200 ng/mL, about 225 ng/ml, about 250 ng/ml, about 300 ng/mL, about 350 ng/ml, about 400 ng/mL, about 450 ng/mL, about 500 ng/ml, but it is not limited thereto. In one specific embodiment, the concentration of thrombopoietin may be about 100 ng/mL. In another specific embodiment, the concentration of thrombopoietin may be about 8 ng/mL. In yet another embodiment, the concentration of thrombopoietin may be about 20 ng/mL. Furthermore, in another embodiment, the concentration of thrombopoietin may be about 50 ng/mL.

In one embodiment, in the foregoing step of culturing a stem cell on the foregoing coating in the presence of a first cytokine combination to differentiate the foregoing stem cell into an immune cell, the concentration of Fms-like tyrosine kinase 3 ligand may be about 1-500 ng/mL, such as about 5-450 ng/mL, about 10-400 ng/mL, about 15-350 ng/ml, about 20-300 ng/mL, about 25-250 ng/mL, about 30-200 ng/mL, about 35-150 ng/ml, about 40-100 ng/mL, about 45-95 ng/mL, about 50-90 ng/ml, about 55-85 ng/ml, about 60-80 ng/ml, about 2 ng/mL, about 3 ng/mL, about 4 ng/ml, about 5 ng/mL, about 6 ng/ml, about 7 ng/ml, about 8 ng/mL, about 9 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/ml, about 75 ng/mL, about 80 ng/mL, about 85 ng/mL, about 90 ng/mL, about 95 ng/mL, about 100 ng/mL, about 125 ng/mL, about 150 ng/mL, about 200 ng/mL, about 225 ng/ml, about 250 ng/ml, about 300 ng/mL, about 350 ng/mL, about 400 ng/mL, about 450 ng/mL, about 500 ng/mL, but it is not limited thereto. In one specific embodiment, the concentration of Fms-like tyrosine kinase 3ligand may be about 100 ng/mL. In another specific embodiment, the concentration of Fms-like tyrosine kinase 3 ligand may be about 8 ng/mL. In yet another embodiment, the concentration of Fms-like tyrosine kinase 3 ligand may be about 20 ng/mL. Furthermore, in another embodiment, the concentration of Fms-like tyrosine kinase 3 ligand may be about 50 ng/mL.

In addition, in one embodiment, in the foregoing step of culturing a stem cell on the foregoing coating in the presence of a first cytokine combination to differentiate the foregoing stem cell into an immune cell, the concentration of interleukin-7 may be about 1-500 ng/ml, such as about 5-450 ng/mL, about 10-400 ng/mL, about 15-350 ng/ml, about 20-300 ng/mL, about 25-250 ng/mL, about 30-200 ng/mL, about 35-150 ng/ml, about 40-100 ng/mL, about 45-95 ng/mL, about 50-90 ng/ml, about 55-85 ng/ml, about 60-80 ng/mL, about 2 ng/mL, about 3 ng/mL, about 4 ng/mL, about 5 ng/mL, about 6 ng/ml, about 7 ng/ml, about 8 ng/ml, about 9 ng/ml, about 10 ng/mL, about 15 ng/ml, about 20 ng/mL, about 25 ng/mL, about 30 ng/mL, about 35 ng/ml, about 40 ng/ml, about 45 ng/ml, about 50 ng/mL, about 55 ng/ml, about 60 ng/ml, about 65 ng/ml, about 70 ng/mL, about 75 ng/ml, about 80 ng/mL, about 85 ng/mL, about 90 ng/mL, about 95 ng/mL, about 100 ng/ml, about 125 ng/mL, about 150 ng/mL, about 200 ng/mL, about 225 ng/ml, about 250 ng/ml, about 300 ng/mL, about 350 ng/mL, about 400 ng/mL, about 450 ng/mL, about 500 ng/mL, but it is not limited thereto. In one specific embodiment, the concentration of interleukin-7 may be about 100 ng/mL. In another specific embodiment, the concentration of interleukin-7 may be about 8 ng/mL. In yet another embodiment, the concentration of interleukin-7 may be about 20 ng/mL. Furthermore, in another embodiment, the concentration of interleukin-7 may be about 50 ng/mL.

In another embodiment, in the foregoing step of culturing a stem cell on the foregoing coating in the presence of a first cytokine combination to differentiate the foregoing stem cell into an immune cell, the concentration of stem cell factor, the concentration of thrombopoietin, the concentration of Fms-like tyrosine kinase 3 ligand and the concentration of interleukin-7 may independently be about 1-500 ng/ml, such as about 5-450 ng/mL, about 10-400 ng/ml, about 15-350 ng/mL, about 20-300 ng/mL, about 25-250 ng/mL, about 30-200 ng/mL, about 35-150 ng/ml, about 40-100 ng/ml, about 45-95 ng/mL, about 50-90 ng/ml, about 55-85 ng/mL, about 60-80 ng/ml, about 2 ng/mL, about 3 ng/mL, about 4 ng/mL, about 5 ng/ml, about 6 ng/ml, about 7 ng/ml, about 8 ng/ml, about 9 ng/mL, about 10 ng/mL, about 15 ng/mL, about 20 ng/ml, about 25 ng/ml, about 30 ng/mL, about 35 ng/mL, about 40 ng/mL, about 45 ng/ml, about 50 ng/ml, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 125 ng/ml, about 150 ng/mL, about 200 ng/mL, about 225 ng/ml, about 250 ng/ml, about 300 ng/ml, about 350 ng/mL, about 400 ng/ml, about 450 ng/mL, about 500 ng/ml, but it is not limited thereto. In one specific embodiment, the concentration of stem cell factor, the concentration of thrombopoietin, the concentration of Fms-like tyrosine kinase 3 ligand and the concentration of interleukin-7 may all be about 100 ng/mL. In another specific embodiment, the concentration of stem cell factor, the concentration of thrombopoietin, the concentration of Fms-like tyrosine kinase 3 ligand and the concentration of interleukin-7 may all be about 8 ng/mL. In yet another embodiment, the concentration of stem cell factor, the concentration of thrombopoietin, the concentration of Fms-like tyrosine kinase 3 ligand and the concentration of interleukin-7 may all be about 20 ng/ml. Furthermore, in another embodiment, the concentration of stem cell factor, the concentration of thrombopoietin, the concentration of Fms-like tyrosine kinase 3 ligand and the concentration of interleukin-7 may all be about 50 ng/mL.

Moreover, in one embodiment, in the foregoing step of culturing a stem cell on the foregoing coating in the presence of a first cytokine combination to differentiate the foregoing stem cell into an immune cell, the foregoing stem cell may be culture with a medium, and the medium mentioned above may comprise, but is not limited to, Iscove's modified Dulbecco's medium (IMDM), a modified Iscove's modified Dulbecco's medium, Roswell Park Memorial Institute (RPMI) medium, etc. In one specific embodiment, the medium mentioned above is a modified Iscove's modified Dulbecco's medium.

In addition, in one embodiment, in the foregoing step of culturing a stem cell on the foregoing coating in the presence of a first cytokine combination to differentiate the foregoing stem cell into an immune cell, the foregoing stem cell may be cultured at about 35-37° C., such as about 35° C., about 35.5° C., about 36° C., about 36.5° C., about 37° C., but it is not limited thereto.

In one embodiment, in the foregoing step of culturing a stem cell on the foregoing coating in the presence of a first cytokine combination to differentiate the foregoing stem cell into an immune cell, the foregoing stem cell may be cultured for about 7-42 days, such as about 10-40 days, about 12-36 days, about 15-30 days, about 7 days, about 8 days, about 9 days, about 10 days, about 11 days, about 12 days, about 14 days, about 18 days, about 20 days, about 21 days, about 22 days, about 25 days, about 28 days, but it is not limited thereto.

In one embodiment, the method for preparing an immune cell differentiated from a stem cell of the present disclosure, in addition to the foregoing step of forming a coating containing a matrix on a surface and the foregoing step of culturing a stem cell on the foregoing coating in the presence of a first cytokine combination to differentiate the foregoing stem cell into an immune cell, before the step of culturing a stem cell on the foregoing coating in the presence of a first cytokine combination to differentiate the foregoing stem cell into an immune cell, may further comprise screening the stem cell mentioned above from the source of the stem cell mentioned above, but it is not limited thereto.

In another embodiment, the method for preparing an immune cell differentiated from a stem cell of the present disclosure, in addition to the foregoing step of forming a coating containing a matrix on a surface and the foregoing step of culturing a stem cell on the foregoing coating in the presence of a first cytokine combination to differentiate the foregoing stem cell into an immune cell, before the step of culturing a stem cell on the foregoing coating in the presence of a first cytokine combination to differentiate the foregoing stem cell into an immune cell, may further comprise expanding the stem cell mentioned above, but it is also not limited thereto.

In yet another embodiment, the method for preparing an immune cell differentiated from a stem cell of the present disclosure, in addition to the foregoing step of forming a coating containing a matrix on a surface and the foregoing step of culturing a stem cell on the foregoing coating in the presence of a first cytokine combination to differentiate the foregoing stem cell into an immune cell, before the step of culturing a stem cell on the foregoing coating in the presence of a first cytokine combination to differentiate the foregoing stem cell into an immune cell, may further comprise, but is not limited to, screening the stem cell mentioned above from the source of the stem cell mentioned above, and expanding the stem cell mentioned above after screening the stem cell mentioned above.

Moreover, a procedure for expanding the aforementioned stem cell may comprise, but is not limited to, culturing the aforementioned stem cell in the presence of a second cytokine combination.

The foregoing second cytokine combination may comprise stem cell factor, thrombopoietin, Fms-like tyrosine kinase 3 ligand (Flt3L), interleukin-3 (IL-3) and interleukin-6 (IL-6), but it is not limited thereto.

In one embodiment, in the procedure of expanding the stem cell mentioned above, the concentration of stem cell factor may be about 1-500 ng/mL, such as about 5-450 ng/ml, about 10-400 ng/mL, about 15-350 ng/mL, about 20-300 ng/mL, about 25-250 ng/mL, about 30-200 ng/mL, about 35-150 ng/mL, about 40-100 ng/ml, about 45-95 ng/ml, about 50-90 ng/mL, about 55-85 ng/mL, about 60-80 ng/mL, about 2 ng/ml, about 3 ng/ml, about 4 ng/mL, about 5 ng/mL, about 6 ng/mL, about 7 ng/mL, about 8 ng/mL, about 9 ng/ml, about 10 ng/mL, about 15 ng/mL, about 20 ng/ml, about 25 ng/ml, about 30 ng/mL, about 35 ng/mL, about 40 ng/mL, about 45 ng/mL, about 50 ng/mL, about 55 ng/mL, about 60 ng/ml, about 65 ng/mL, about 70 ng/mL, about 75 ng/mL, about 80 ng/ml, about 85 ng/ml, about 90 ng/mL, about 95 ng/mL, about 100 ng/mL, about 125 ng/mL, about 150 ng/mL, about 200 ng/mL, about 225 ng/mL, about 250 ng/mL, about 300 ng/mL, about 350 ng/ml, about 400 ng/mL, about 450 ng/ml, about 500 ng/ml, but it is not limited thereto. In one specific embodiment, the concentration of stem cell factor may be about 100 ng/ml. In another specific embodiment, the concentration of stem cell factor may be about 8 ng/ml. In yet another embodiment, the concentration of stem cell factor may be about 20 ng/mL. Moreover, in another embodiment, the concentration of stem cell factor may be about 50 ng/mL.

In one embodiment, in the procedure of expanding the stem cell mentioned above, the concentration of thrombopoietin may be about 1-500 ng/mL, such as about 5-450 ng/mL, about 10-400 ng/mL, about 15-350 ng/mL, about 20-300 ng/mL, about 25-250 ng/ml, about 30-200 ng/mL, about 35-150 ng/mL, about 40-100 ng/ml, about 45-95 ng/ml, about 50-90 ng/mL, about 55-85 ng/mL, about 60-80 ng/mL, about 2 ng/mL, about 3 ng/ml, about 4 ng/mL, about 5 ng/mL, about 6 ng/mL, about 7 ng/mL, about 8 ng/mL, about 9 ng/ml, about 10 ng/ml, about 15 ng/ml, about 20 ng/mL, about 25 ng/mL, about 30 ng/ml, about 35 ng/mL, about 40 ng/mL, about 45 ng/mL, about 50 ng/mL, about 55 ng/mL, about 60 ng/ml, about 65 ng/ml, about 70 ng/mL, about 75 ng/mL, about 80 ng/mL, about 85 ng/ml, about 90 ng/mL, about 95 ng/mL, about 100 ng/mL, about 125 ng/mL, about 150 ng/ml, about 200 ng/mL, about 225 ng/mL, about 250 ng/mL, about 300 ng/mL, about 350 ng/ml, about 400 ng/mL, about 450 ng/mL, about 500 ng/mL, but it is not limited thereto. In one specific embodiment, the concentration of thrombopoietin may be about 100 ng/mL. In another specific embodiment, the concentration of thrombopoietin may be about 8 ng/ml. In yet another embodiment, the concentration of thrombopoietin may be about 20 ng/ml. Furthermore, in another embodiment, the concentration of thrombopoietin may be about 50 ng/mL.

Moreover, in one embodiment, in the procedure of expanding the stem cell mentioned above, the concentration of Fms-like tyrosine kinase 3 ligand may be about 1-500 ng/mL, such as about 5-450 ng/mL, about 10-400 ng/mL, about 15-350 ng/ml, about 20-300 ng/mL, about 25-250 ng/ml, about 30-200 ng/mL, about 35-150 ng/ml, about 40-100 ng/mL, about 45-95 ng/mL, about 50-90 ng/mL, about 55-85 ng/ml, about 60-80 ng/mL, about 2 ng/ml, about 3 ng/mL, about 4 ng/mL, about 5 ng/mL, about 6 ng/ml, about 7 ng/ml, about 8 ng/mL, about 9 ng/mL, about 10 ng/mL, about 15 ng/ml, about 20 ng/mL, about 25 ng/mL, about 30 ng/mL, about 35 ng/mL, about 40 ng/ml, about 45 ng/mL, about 50 ng/ml, about 55 ng/ml, about 60 ng/mL, about 65 ng/mL, about 70 ng/mL, about 75 ng/ml, about 80 ng/mL, about 85 ng/mL, about 90 ng/mL, about 95 ng/ml, about 100 ng/mL, about 125 ng/mL, about 150 ng/mL, about 200 ng/mL, about 225 ng/mL, about 250 ng/mL, about 300 ng/mL, about 350 ng/mL, about 400 ng/mL, about 450 ng/mL, about 500 ng/ml, but it is not limited thereto. In one specific embodiment, the concentration of Fms-like tyrosine kinase 3 ligand may be about 100 ng/mL. In another specific embodiment, the concentration of Fms-like tyrosine kinase 3 ligand may be about 8 ng/mL. In yet another embodiment, the concentration of Fms-like tyrosine kinase 3 ligand may be about 20 ng/mL. Furthermore, in another embodiment, the concentration of Fms-like tyrosine kinase 3 ligand may be about 50 ng/mL.

Furthermore, in one embodiment, in the procedure of expanding the stem cell mentioned above, the concentration of interleukin-3 may be about 1-500 ng/mL, such as about 5-450 ng/mL, about 10-400 ng/mL, about 15-350 ng/ml, about 20-300 ng/ml, about 25-250 ng/mL, about 30-200 ng/ml, about 35-150 ng/mL, about 40-100 ng/ml, about 45-95 ng/ml, about 50-90 ng/mL, about 55-85 ng/ml, about 60-80 ng/mL, about 2 ng/ml, about 3 ng/mL, about 4 ng/mL, about 5 ng/mL, about 6 ng/mL, about 7 ng/ml, about 8 ng/ml, about 9 ng/mL, about 10 ng/mL, about 15 ng/ml, about 20 ng/mL, about 25 ng/ml, about 30 ng/mL, about 35 ng/ml, about 40 ng/mL, about 45 ng/ml, about 50 ng/ml, about 55 ng/mL, about 60 ng/mL, about 65 ng/mL, about 70 ng/mL, about 75 ng/ml, about 80 ng/ml, about 85 ng/mL, about 90 ng/mL, about 95 ng/mL, about 100 ng/ml, about 125 ng/ml, about 150 ng/mL, about 200 ng/mL, about 225 ng/mL, about 250 ng/mL, about 300 ng/ml, about 350 ng/mL, about 400 ng/mL, about 450 ng/mL, about 500 ng/mL, but it is not limited thereto. In one specific embodiment, the concentration of interleukin-3 may be about 100 ng/ml. In another specific embodiment, the concentration of interleukin-3 may be about 8 ng/ml. In yet another embodiment, the concentration of interleukin-3 may be about 20 ng/ml. Moreover, in another embodiment, the concentration of interleukin-3 may be about 50 ng/mL.

In addition, in one embodiment, in the procedure of expanding the stem cell mentioned above, the concentration of interleukin-6 may be about 1-500 ng/ml, such as about 5-450 ng/mL, about 10-400 ng/ml, about 15-350 ng/mL, about 20-300 ng/ml, about 25-250 ng/mL, about 30-200 ng/mL, about 35-150 ng/mL, about 40-100 ng/mL, about 45-95 ng/mL, about 50-90 ng/mL, about 55-85 ng/mL, about 60-80 ng/mL, about 2 ng/mL, about 3 ng/mL, about 4 ng/mL, about 5 ng/ml, about 6 ng/ml, about 7 ng/ml, about 8 ng/ml, about 9 ng/mL, about 10 ng/mL, about 15 ng/mL, about 20 ng/ml, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/ml, about 75 ng/mL, about 80 ng/ml, about 85 ng/mL, about 90 ng/mL, about 95 ng/mL, about 100 ng/ml, about 125 ng/ml, about 150 ng/mL, about 200 ng/mL, about 225 ng/mL, about 250 ng/ml, about 300 ng/ml, about 350 ng/mL, about 400 ng/mL, about 450 ng/mL, about 500 ng/mL, but it is not limited thereto. In one specific embodiment, the concentration of interleukin-6 may be about 100 ng/ml. In another specific embodiment, the concentration of interleukin-6 may be about 8 ng/ml. In yet another embodiment, the concentration of interleukin-6 may be about 20 ng/mL. Moreover, in another embodiment, the concentration of interleukin-6 may be about 50 ng/mL.

In another embodiment, in the procedure of expanding the stem cell mentioned above, the concentration of stem cell factor, the concentration of thrombopoietin, the concentration of Fms-like tyrosine kinase 3 ligand, the concentration of interleukin-3 and the concentration of interleukin-6 may independently about 1-500 ng/mL, such as about 5-450 ng/mL, about 10-400 ng/mL, about 15-350 ng/mL, about 20-300 ng/mL, about 25-250 ng/ml, about 30-200 ng/mL, about 35-150 ng/ml, about 40-100 ng/mL, about 45-95 ng/ml, about 50-90 ng/mL, about 55-85 ng/mL, about 60-80 ng/mL, about 2 ng/mL, about 3 ng/ml, about 4 ng/mL, about 5 ng/mL, about 6 ng/mL, about 7 ng/mL, about 8 ng/ml, about 9 ng/ml, about 10 ng/mL, about 15 ng/mL, about 20 ng/mL, about 25 ng/ml, about 30 ng/ml, about 35 ng/ml, about 40 ng/mL, about 45 ng/ml, about 50 ng/ml, about 55 ng/mL, about 60 ng/mL, about 65 ng/mL, about 70 ng/mL, about 75 ng/ml, about 80 ng/ml, about 85 ng/ml, about 90 ng/mL, about 95 ng/mL, about 100 ng/mL, about 125 ng/ml, about 150 ng/ml, about 200 ng/mL, about 225 ng/mL, about 250 ng/mL, about 300 ng/ml, about 350 ng/mL, about 400 ng/mL, about 450 ng/ml, about 500 ng/ml, but they are not limited thereto. In one specific embodiment, the concentration of stem cell factor, the concentration of thrombopoietin, the concentration of Fms-like tyrosine kinase 3 ligand, the concentration of interleukin-3 and the concentration of interleukin-6 may all be about 100 ng/mL. In another specific embodiment, the concentration of stem cell factor, the concentration of thrombopoietin, the concentration of Fms-like tyrosine kinase 3 ligand, the concentration of interleukin-3 and the concentration of interleukin-6 may all be about 8 ng/mL. In yet another embodiment, the concentration of stem cell factor, the concentration of thrombopoietin, the concentration of Fms-like tyrosine kinase 3 ligand, the concentration of interleukin-3 and the concentration of interleukin-6 may all be about 20 ng/mL. Moreover, in another embodiment, the concentration of stem cell factor, the concentration of thrombopoietin, the concentration of Fms-like tyrosine kinase 3 ligand, the concentration of interleukin-3 and the concentration of interleukin-6 may all be about 50 ng/mL.

Furthermore, in one embodiment, in the procedure of expanding the stem cell mentioned above, the stem cell mentioned above may be cultured with another medium, and the other medium may comprise, but is not limited to, Iscove's modified Dulbecco's medium (IMDM), a modified Iscove's modified Dulbecco's medium, Roswell Park Memorial Institute (RPMI) medium, etc. In one specific embodiment, the other medium mentioned above is a modified Iscove's modified Dulbecco's medium.

In addition, in the procedure of expanding the stem cell mentioned above, the stem cell mentioned above can be cultured at about 35-37° C., such as about 35° C., about 35.5° C., about 36° C., about 36.5° C., about 37° C., but it is not limited thereto.

Moreover, in the procedure of expanding the stem cell mentioned above, the stem cell mentioned above can be cultured for about 5-7 days, such as about 5 days, about 5.5 days, about 6 days, about 6.5 days, about 7 days, but it is not limited thereto.

In addition, based on the foregoing, the present disclosure may also provide a kit for preparing an immune cell differentiated from a stem cell.

By using the kit for preparing an immune cell differentiated from a stem cell of the present disclosure, a stem cell can be effectively differentiated into an immune cell, especially a T cell.

A source of the stem cell mentioned above may comprise umbilical cord blood or an induced pluripotent stem cell (iPSC), but it is not limited thereto. In addition, the stem cell mentioned above may be CD34 positive, but it is also not limited thereto. In one embodiment, the stem cell mentioned above is a hematopoietic stem cell (HSC). Furthermore, in one embodiment, the immune cell mentioned above may comprise a γδ T cell, a NK cell, etc., but it is not limited thereto.

The kit for preparing an immune cell differentiated from a stem cell of the present disclosure may comprise, but is not limited to, a coating formation sub-kit for forming a coating on a surface, and a stem cell differentiation sub-kit.

The coating formation sub-kit mentioned above may comprise, but is not limited to, a matrix component for forming a coating formation solution with a solvent. The matrix component may comprise vascular cell adhesion molecule (VCAM), intercellular adhesion molecule (ICAM) and delta-like ligand 4 (DLL4), but it is not limited thereto. Moreover, the coating formation solution mentioned above may be used to coat the surface mentioned above to form the coating mentioned above on the surface mentioned above. The solvent mentioned above may comprise, but is not limited to, phosphate buffered saline (PBS).

A manner for forming a coating by the coating formation solution is not particularly limited, as long as the coating formation solution can form a coating containing the aforementioned matrix on the aforementioned surface, ultimately. In one embodiment, the coating formation solution mentioned above is brought into contact with the surface mentioned above, and the coating formation solution mentioned above is covered on the surface mentioned above, so as to form a coating containing the matrix mentioned above on the surface mentioned above.

In one embodiment, in the foregoing matrix component of the foregoing coating formation sub-kit, or in the foregoing coating formation solution or the foregoing coating, the content ratio of vascular cell adhesion molecule, intercellular adhesion molecule and delta-like ligand 4 may be about 0.01-50:0.01-50:0.01-50, such as about 0.05-50:0.05-50:0.05-50, about 0.1-45:0.1-45:0.1-45, about 0.5-40:0.5-40:0.5-40, about 1-35:1-35:1-35, about 2-30:2-30:2-30, about 2.5-25:2.5-25:2.5-25, about 4-20:4-20:4-20, about 5-15:5-15:5-15 [symbol] 0.1-10:0.1-10:0.1-10, about 1:1:1, about 1:2:1, about 1:1:2, about 1:2:2, about 2:1:1, about 2:2:1, about 1:3:1, about 1:1:3, about 1:3:3, about 3:1:1, about 3:3:1, about 1:4:1, about 1:1:4, about 1:4:4, about 4:1:1, about 4:4:1, about 1:5:1, about 1:1:5, about 1:5:5, about 5:1:1, about 5:5:1, about 1:6:1, about 1:1:6, about 1:6:6, about 6:1:1, about 6:6:1, about 1:7:1, about 1:1:7, about 1:7:7, about 7:1:1, about 7:7:1, about 1:8:1, about 1:1:8, about 1:8:8, about 8:1:1, about 8:8:1, about 1:9:1, about 1:1:9, about 1:9:9, about 9:1:1, about 9:9:1, about 1:10:1, about 1:1:10, about 1:10:10, about 10:1:1, about 10:10:1, about 1:50:1, about 1:1:50, about 1:50:50, about 50:1:1, about 50:50:1, about 1:100:1, about 1:1:100, about 1:100:100, about 100:1:1, about 100:100:1, but it is not limited thereto. In one specific embodiment, in the foregoing matrix component of the foregoing coating formation sub-kit, or in the foregoing coating formation solution or the foregoing coating, the content ratio of vascular cell adhesion molecule, intercellular adhesion molecule and delta-like ligand 4 may be about 1:1:1.

With regard to relevant descriptions for the respective concentrations of vascular cell adhesion molecule, intercellular adhesion molecule and delta-like ligand 4 in the coating formation solution mentioned above, please refer to respective descriptions related to the concentrations of vascular cell adhesion molecule, intercellular adhesion molecule and delta-like ligand 4 in the coating formation solution in the related descriptions for the method for preparing an immune cell differentiated from a stem cell of the present disclosure above, and thus will not be repeated here.

In one embodiment, vascular cell adhesion molecule, intercellular adhesion molecule and delta-like ligand 4 in the above-mentioned matrix component of the above-mentioned coating formation sub-kit may be packaged in the same container. In one specific embodiment of this embodiment, by mixing all the contents in the container of packaging vascular cell adhesion molecule, intercellular adhesion molecule and delta-like ligand 4 with a specified amount of the above-mentioned solvent, the above-mentioned coating formation solution can be formed. In another specific embodiment of this embodiment, by taking a specified amount of the contents in the container of packaging vascular cell adhesion molecule, intercellular adhesion molecule and delta-like ligand 4 and mixing them with a specified amount of the above-mentioned solvent, the above-mentioned coating formation solution can be formed.

In another embodiment, vascular cell adhesion molecule, intercellular adhesion molecule and delta-like ligand 4 in the above-mentioned matrix component of the above-mentioned coating formation sub-kit may be respectively packaged in different containers. In one specific embodiment of this embodiment, by mixing all the contents in the container of packaging vascular cell adhesion molecule, all the contents in the container of packaging intercellular adhesion molecule and all the contents in the container of packaging delta-like ligand 4 with a specified amount of the above-mentioned solvent, the above-mentioned coating formation solution can be formed. In another specific embodiment of this embodiment, by taking a specified amount of the contents in the container of packaging vascular cell adhesion molecule, taking a specified amount of the contents in the container of packaging intercellular adhesion molecule and taking a specified amount of the contents in the container of packaging delta-like ligand 4, and mixing them with a specified amount of the above-mentioned solvent, the above-mentioned coating formation solution can be formed.

Moreover, in one embodiment, the foregoing coating formation sub-kit may further comprise, but is not limited to, the foregoing solvent to form the foregoing coating formation solution with the foregoing matrix component. The foregoing solvent may comprise, but is not limited to, phosphate buffered saline (PBS).

In one specific embodiment of this embodiment, the above-mentioned matrix component and the above-mentioned solvent may be packaged in the same container to form the above-mentioned coating formation solution.

In another specific embodiment of this embodiment, the above-mentioned matrix component and the above-mentioned solvent may be packaged in different containers. In this specific embodiment, when using the kit of the present disclosure, all the contents of the container of packaging the above-mentioned solvent are added to the container of packaging the above-mentioned matrix component to mix with the above-mentioned matrix component to form the above-mentioned coating formation solution, or all the contents of the container of packaging the above-mentioned matrix component are added to the container of packaging the above-mentioned solvent to mix with the above-mentioned solvent to form the above-mentioned coating formation solution.

The stem cell differentiation sub-kit mentioned above in the kit for preparing an immune cell differentiated from a stem cell of the present disclosure may comprise, but is not limited to, a first cytokine combination for adding to a medium to form a stem cell differentiation medium. The medium mentioned above may comprise, but is not limited to, Iscove's modified Dulbecco's medium (IMDM), a modified Iscove's modified Dulbecco's medium, Roswell Park Memorial Institute (RPMI) medium, etc. In one specific embodiment, the medium mentioned above is a modified Iscove's modified Dulbecco's medium.

The foregoing first cytokine combination may comprise stem cell factor, thrombopoietin, Fms-like tyrosine kinase 3 ligand and interleukin-7, but it is not limited thereto.

In one embodiment, in the foregoing first cytokine combination of the foregoing stem cell differentiation sub-kit, the content ratio of stem cell factor, thrombopoietin, Fms-like tyrosine kinase 3 ligand and interleukin-7 may be about 1-50:1-50:1-50:1-50, such as about 1-45:1-45:1-45:1-45, about 2-40:2-40:2-40:2-40, about 3-35:3-35:3-35:3-35, about 4-30:4-30:4-30:4-30, about 5-25:5-25:5-25:5-25, about 6-20:6-20:6-20:6-20, about 7-15:7-15:7-15:7-15, about 1:1:1:1, about 1:2:1:1, about 1:1:2:1, about 1:1:1:2, about 2:1:1:1, about 1:2:2:1, about 2:2:1:1, about 1:1:2:2, about 2:1:2:1, about 2:1:1:2, about 1:2:1:2, about 1:2:2:2, about 2:1:2:2, about 2:2:1:2, about 2:2:2:1, about 1:3:1:1, about 1:1:3:1, about 1:1:1:3, about 3:1:1:1, about 1:3:3:1, about 3:3:1:1, about 1:1:3:3, about 3:1:3:1, about 3:1:1:3, about 1:3:1:3, about 1:3:3:3, about 3:1:3:3, about 3:3:1:3, about 3:3:3:1, about 1:4:1:1, about 1:1:4:1, about 1:1:1:4, about 4:1:1:1, about 1:4:4:1, about 4:4:1:1, about 1:1:4:4, about 4:1:4:1, about 4:1:1:4, about 1:4:1:4, about 1:4:4:4, about 4:1:4:4, about 4:4:1:4, about 4:4:4:1, about 1:5:1:1, about 1:1:5:1, about 1:1:1:5, about 5:1:1:1, about 1:5:5:1, about 5:5:1:1, about 1:1:5:5, about 5:1:5:1, about 5:1:1:5, about 1:5:1:5, about 1:5:5:5, about 5:1:5:5, about 5:5:1:5, about 5:5:5:1, about 1:6:1:1, about 1:1:6:1, about 1:1:1:6, about 6:1:1:1, about 1:6:6:1, about 6:6:1:1, about 1:1:6:6, about 6:1:6:1, about 6:1:1:6, about 1:6:1:6, about 1:6:6:6, about 6:1:6:6, about 6:6:1:6, about 6:6:6:1, about 1:7:1:1, about 1:1:7:1, about 1:1:1:7, about 7:1:1:1, about 1:7:7:1, about 7:7:1:1, about 1:1:7:7, about 7:1:7:1, about 7:1:1:7, about 1:7:1:7, about 1:7:7:7, about 7:1:7:7, about 7:7:1:7, about 7:7:7:1, about 1:8:1:1, about 1:1:8:1, about 1:1:1:8, about 8:1:1:1, about 1:8:8:1, about 8:8:1:1, about 1:1:8:8, about 8:1:8:1, about 8:1:1:8, about 1:8:1:8, about 1:8:8:8, about 8:1:8:8, about 8:8:1:8, about 8:8:8:1, about 1:9:1:1, about 1:1:9:1, about 1:1:1:9, about 9:1:1:1, about 1:9:9:1, about 9:9:1:1, about 1:1:9:9, about 9:1:9:1, about 9:1:1:9, about 1:9:1:9, about 1:9:9:9, about 9:1:9:9, about 9:9:1:9, about 9:9:9:1, about 1:10:1:1, about 1:1:10:1, about 1:1:1:10, about 10:1:1:1, about 1:10:10:1, about 10:10:1:1, about 1:1:10:10, about 10:1:10:1, about 10:1:1:10, about 1:10:1:10, about 1:10:10:10, about 10:1:10:10, about 10:10:1:10, about 10:10:10:1, about 1:50:1:1, about 1:1:50:1, about 1:1:1:50, about 50:1:1:1, about 1:50:50:1, about 50:50:1:1, about 1:1:50:50, about 50:1:50:1, about 50:1:1:10, about 1:50:1:50, about 1:50:50:50, about 50:1:50:50, about 50:50:1:50, about 50:50:50:1, but it is not limited thereto. In one specific embodiment, in the foregoing first cytokine combination of the foregoing stem cell differentiation sub-kit, the content ratio of stem cell factor, thrombopoietin, Fms-like tyrosine kinase 3 ligand and interleukin-7 may be about 1:1:1:1.

With regard to relevant descriptions for the respective concentrations of stem cell factor, thrombopoietin, Fms-like tyrosine kinase 3 ligand and interleukin-7 in the stem cell differentiation medium mentioned above, please refer to respective descriptions related to the concentrations of stem cell factor, thrombopoietin, Fms-like tyrosine kinase 3 ligand and interleukin-7 in the foregoing step of culturing a stem cell on the foregoing coating in the presence of a first cytokine combination to differentiate the foregoing stem cell into an immune cell in the related descriptions for the method for preparing an immune cell differentiated from a stem cell of the present disclosure above, and thus will not be repeated here.

In one embodiment, stem cell factor, thrombopoietin, Fms-like tyrosine kinase 3 ligand and interleukin-7 in the first cytokine combination mentioned above of the stem cell differentiation sub-kit mentioned above may be packaged in the same container. In one specific embodiment of this embodiment, by mixing all the contents in the container of packaging stem cell factor, thrombopoietin, Fms-like tyrosine kinase 3 ligand and interleukin-7 with a specified amount of the medium mentioned above, the stem cell differentiation medium mentioned above can be formed. In another specific embodiment of this embodiment, by taking a specified amount of the contents in the container of packaging stem cell factor, thrombopoietin, Fms-like tyrosine kinase 3 ligand and interleukin-7 and mixing them with a specified amount of the medium mentioned above, the stem cell differentiation medium mentioned above can be formed.

In another embodiment, stem cell factor, thrombopoietin, Fms-like tyrosine kinase 3 ligand and interleukin-7 in the first cytokine combination mentioned above of the stem cell differentiation sub-kit mentioned above may be respectively packaged in different containers. In one specific embodiment of this embodiment, by mixing all the contents in the container of packaging stem cell factor, all the contents in the container of packaging thrombopoietin, all the contents in the container of packaging Fms-like tyrosine kinase 3 ligand and all the contents in the container of packaging interleukin-7 with a specified amount of the medium mentioned above, the stem cell differentiation medium mentioned above can be formed. In another specific embodiment of this embodiment, by taking a specified amount of the contents in the container of packaging stem cell factor, taking a specified amount of the contents in the container of packaging thrombopoietin, taking a specified amount of the contents in the container of packaging Fms-like tyrosine kinase 3 ligand and taking a specified amount of the contents in the container of packaging interleukin-7, and mixing them with a specified amount of the medium mentioned above, the stem cell differentiation medium mentioned above can be formed.

Furthermore, in one embodiment, the aforementioned stem cell differentiation sub-kit may further comprise, but is not limited to, the aforementioned medium to form the aforementioned stem cell differentiation medium with the aforementioned first cytokine combination. The aforementioned medium may comprise but is not limited to, Iscove's modified Dulbecco's medium (IMDM), a modified Iscove's modified Dulbecco's medium, Roswell Park Memorial Institute (RPMI) medium, etc. In one specific embodiment, the aforementioned medium is a modified Iscove's modified Dulbecco's medium.

In one specific embodiment of this embodiment, the above-mentioned first cytokine combination and the above-mentioned medium may be packaged in the same container to become the above-mentioned stem cell differentiation medium.

In another specific embodiment of this embodiment, the above-mentioned first cytokine combination and the above-mentioned medium may be packaged in different containers. In this specific embodiment, when using the kit of the present disclosure, all the contents of the container of packaging the above-mentioned first cytokine combination are added to the container of packaging the above-mentioned medium to mix with the above-mentioned medium to form the above-mentioned stem cell differentiation medium.

In addition, the foregoing kit for preparing an immune cell differentiated from a stem cell of the present disclosure may further comprise, but is not limited to a stem cell expansion sub-kit which is used for expanding the stem cell.

The foregoing stem cell expansion sub-kit in the kit for preparing an immune cell differentiated from a stem cell of the present disclosure may comprise, but is not limited to, a second cytokine combination for adding to another medium to form a stem cell expansion medium. The foregoing other medium may comprise, but is not limited to, Iscove's modified Dulbecco's medium (IMDM), a modified Iscove's modified Dulbecco's medium, Roswell Park Memorial Institute (RPMI) medium, etc. In one specific embodiment, the foregoing other medium is a modified Iscove's modified Dulbecco's medium.

The second cytokine combination mentioned above may comprise stem cell factor, thrombopoietin, Fms-like tyrosine kinase 3 ligand (Flt3L), interleukin-3 (IL-3) and interleukin-6 (IL-6), but it is not limited thereto.

In one embodiment, in the second cytokine combination mentioned above of the stem cell expansion sub-kit mentioned above, the content ratio of stem cell factor, thrombopoietin, Fms-like tyrosine kinase 3 ligand, interleukin-3 and interleukin-6 may be about 1-50:1-50:1-50:1-50:1-50, such as about 1-45:1-45:1-45:1-45:1-45, about 2-40:2-40:2-40:2-40:2-40, about 3-35:3-35:3-35:3-35:3-35, about 4-30:4-30:4-30:4-30:4-30, about 5-25:5-25:5-25:5-25:5-25, about 6-20:6-20:6-20:6-20:6-20, about 7-15:7-15:7-15:7-15:7-15, about 1:1:1:1:1, about 1:2:1:1:1, about 1:1:2:1:1, about 1:1:1:2:1, about 1:1:1:1:2, about 2:1:1:1:1, about 1:2:2:1:1, about 2:2:1:1:1, about 1:1:2:2:1, about 2:1:2:1:1, about 2:1:1:2:1, about 1:2:1:2:1, about 1:1:1:2:2, about 1:2:2:2:1, about 2:1:2:2:1, about 2:2:1:2:1, about 2:2:2:1:1, about 1:1:2:2:2, about 1:3:1:1:1, about 1:1:3:1:1, about 1:1:1:3:1, about 1:1:1:1:3, about 3:1:1:1:1, about 1:3:3:1:1, about 3:3:1:1:1, about 1:1:3:3:1, about 3:1:3:1:1, about 3:1:1:3:1, about 1:3:1:3:1, about 1:1:1:3:3, about 1:3:3:3:1, about 3:1:3:3:1, about 3:3:1:3:1, about 3:3:3:1:1, about 1:1:3:3:3, about 1:4:1:1:1, about 1:1:4:1:1, about 1:1:1:4:1, about 1:1:1:1:4, about 4:1:1:1:1, about 1:4:4:1:1, about 4:4:1:1:1, about 1:1:4:4:1, about 4:1:4:1:1, about 4:1:1:4:1, about 1:4:1:4:1, about 1:1:1:4:4, about 1:4:4:4:1, about 4:1:4:4:1, about 4:4:1:4:1, about 4:4:4:1:1, about 1:1:4:4:4, about 1:5:1:1:1, about 1:1:5:1:1, about 1:1:1:5:1, about 1:1:1:1:5, about 5:1:1:1:1, about 1:5:5:1:1, about 5:5:1:1:1, about 1:1:5:5:1, about 5:1:5:1:1, about 5:1:1:5:1, about 1:5:1:5:1, about 1:1:1:5:5, about 1:5:5:5:1, about 5:1:5:5:1, about 5:5:1:5:1, about 5:5:5:1:1, about 1:1:5:5:5, about 1:6:1:1:1, about 1:1:6:1:1, about 1:1:1:6:1, about 1:1:1:1:6, about 6:1:1:1:1, about 1:6:6:1:1, about 6:6:1:1:1, about 1:1:6:6:1, about 6:1:6:1:1, about 6:1:1:6:1, about 1:6:1:6:1, about 1:1:1:6:6, about 1:6:6:6:1, about 6:1:6:6:1, about 6:6:1:6:1, about 6:6:6:1:1, about 1:1:6:6:6, about 1:7:1:1:1, about 1:1:7:1:1, about 1:1:1:7:1, about 1:1:1:1:7, about 7:1:1:1:1, about 1:7:7:1:1, about 7:7:1:1:1, about 1:1:7:7:1, about 7:1:7:1:1, about 7:1:1:7:1, about 1:7:1:7:1, about 1:1:1:7:7, about 1:7:7:7:1, about 7:1:7:7:1, about 7:7:1:7:1, about 7:7:7:1:1, about 1:1:7:7:7, about 1:8:1:1:1, about 1:1:8:1:1, about 1:1:1:8:1, about 1:1:1:1:8, about 8:1:1:1:1, about 1:8:8:1:1, about 8:8:1:1:1, about 1:1:8:8:1, about 8:1:8:1:1, about 8:1:1:8:1, about 1:8:1:8:1, about 1:1:1:8:8, about 1:8:8:8:1, about 8:1:8:8:1, about 8:8:1:8:1, about 8:8:8:1:1, about 1:1:8:8:8, about 1:9:1:1:1, about 1:1:9:1:1, about 1:1:1:9:1, about 1:1:1:1:9, about 9:1:1:1:1, about 1:9:9:1:1, about 9:9:1:1:1, about 1:1:9:9:1, about 9:1:9:1:1, about 9:1:1:9:1, about 1:9:1:9:1, about 1:1:1:9:9, about 1:9:9:9:1, about 9:1:9:9:1, about 9:9:1:9:1, about 9:9:9:1:1, about 1:1:9:9:9, about 1:10:1:1:1, about 1:1:10:1:1, about 1:1:1:10:1, about 1:1:1:1:10, about 10:1:1:1:1, about 1:10:10:1:1, about 10:10:1:1:1, about 1:1:10:10:1, about 10:1:10:1:1, about 10:1:1:10:1, about 1:10:1:10:1, about 1:1:1:10:10, about 1:10:10:10:1, about 10:1:10:10:1, about 10:10:1:10:1, about 10:10:10:1:1, about 1:1:10:10:10, about 1:50:1:1:1, about 1:1:50:1:1, about 1:1:1:50:1, about 1:1:1:1:50, about 50:1:1:1:1, about 1:50:50:1:1, about 50:50:1:1:1, about 1:1:50:50:1, about 50:1:50:1:1, about 50:1:1:50:1, about 1:50:1:50:1, about 1:1:1:50:50, about 1:50:50:50:1, about 50:1:50:50:1, about 50:50:1:50:1, about 50:50:50:1:1, about 1:1:50:50:50, but it is not limited there to. In one specific embodiment, in the first cytokine combination mentioned above of the stem cell differentiation sub-kit mentioned above, the content ratio of stem cell factor, thrombopoietin, Fms-like tyrosine kinase 3 ligand, interleukin-3 and interleukin-6 may be about 1:1:1:1:1.

With regard to relevant descriptions for the respective concentrations of stem cell factor, thrombopoietin, Fms-like tyrosine kinase 3 ligand, interleukin-3 and interleukin-6 in the stem cell expansion medium mentioned above, please refer to respective descriptions related to the concentrations of stem cell factor, thrombopoietin, Fms-like tyrosine kinase 3 ligand, interleukin-3 and interleukin-6 in the foregoing procedure of expanding the stem cell in the related descriptions for the method for preparing an immune cell differentiated from a stem cell of the present disclosure above, and thus will not be repeated here.

In one embodiment, stem cell factor, thrombopoietin, Fms-like tyrosine kinase 3 ligand, interleukin-3 and interleukin-6 in the second cytokine combination mentioned above of the stem cell expansion sub-kit mentioned above may be packaged in the same container. In one specific embodiment of this embodiment, by mixing all the contents in the container of packaging stem cell factor, thrombopoietin, Fms-like tyrosine kinase 3 ligand, interleukin-3 and interleukin-6 with a specified amount of the medium mentioned above, the stem cell expansion medium mentioned above can be formed. In another specific embodiment of this embodiment, by taking a specified amount of the contents in the container of packaging stem cell factor, thrombopoietin, Fms-like tyrosine kinase 3 ligand, interleukin-3 and interleukin-6 and mixing them with a specified amount of the other medium mentioned above, the stem cell expansion medium mentioned above can be formed.

In another embodiment, stem cell factor, thrombopoietin, Fms-like tyrosine kinase 3 ligand, interleukin-3 and interleukin-6 in the first cytokine combination mentioned above of the stem cell expansion sub-kit mentioned above may be respectively packaged in different containers. In one specific embodiment of this embodiment, by mixing all the contents in the container of packaging stem cell factor, all the contents in the container of packaging thrombopoietin, all the contents in the container of packaging Fms-like tyrosine kinase 3 ligand, all the contents in the container of packaging interleukin-3 and all the contents in the container of packaging interleukin-6 with a specified amount of the medium mentioned above, the stem cell expansion medium mentioned above can be formed. In another specific embodiment of this embodiment, by taking a specified amount of the contents in the container of packaging stem cell factor, taking a specified amount of the contents in the container of packaging thrombopoietin, taking a specified amount of the contents in the container of packaging Fms-like tyrosine kinase 3 ligand, taking a specified amount of the contents in the container of packaging interleukin-3 and taking a specified amount of the contents in the container of packaging interleukin-6, and mixing them with a specified amount of the other medium mentioned above, the stem cell expansion medium mentioned above can be formed.

In one embodiment, the aforementioned stem cell expansion sub-kit may further comprise, but is not limited to, the aforementioned other medium to form the aforementioned stem cell expansion medium with the aforementioned second cytokine combination. The aforementioned other medium may comprise but is not limited to, Iscove's modified Dulbecco's medium (IMDM), a modified Iscove's modified Dulbecco's medium, Roswell Park Memorial Institute (RPMI) medium, etc. In one specific embodiment, the aforementioned other medium is a modified Iscove's modified Dulbecco's medium.

In one specific embodiment of this embodiment, the above-mentioned second cytokine combination and the above-mentioned other medium may be packaged in the same container to become the above-mentioned stem cell expansion medium.

In addition, in another specific embodiment of this embodiment, the above-mentioned second cytokine combination and the above-mentioned other medium may be packaged in different containers. In this specific embodiment, when using the kit of the present disclosure, all the contents of the container of packaging the above-mentioned second cytokine combination are added to the container of packaging the above-mentioned other medium to mix with the above-mentioned other medium to form the above-mentioned stem cell expansion medium.

### EXAMPLES

### Example 1

### Isolation of High Purity Hematopoietic Stem Cells (HSCs) From Umbilical Cord Blood

Isolation of hematopoietic stem cells (CD34 positive) from umbilical cord blood of a donor was performed by using the EasySep™ Human Cord Blood CD34 Positive Selection Kit II (Manufacturer: STEMCELL Technologies).

First, the CD34 positive cell pre-enrichment reagent (RosetteSep™ Cord Blood CD34 Pre-Enrichment Cocktail II) in the kit was added to the umbilical cord blood of the donor to make red blood cells and non-CD34 cells to form immunorosettes and after density gradient centrifugation, the interface layer rich in CD34-positive cells was collected. Next, the CD34 binding reagent (EasySep™ Human CD34 Positive Selection Cocktail) and magnetic particles (EasySep™ Dextran RapidSpheres™) in the kit were added to the collected interface layer to form a mixture. After that, a separation tube containing this mixture was placed on a magnetic stand (EasySep™ EasyStand™) (Manufacturer: STEMCELL Technologies), so that the magnetic stand could absorb the CD34 positive stem cells bound to the magnetic particles in the mixture in the separation tube, and the non-target cells could be poured out directly from the separation tube, while the CD34 positive stem cells remained in the separation tube on the magnetic stand. The separation tube was removed from the magnetic holder and the cells therein were collected.

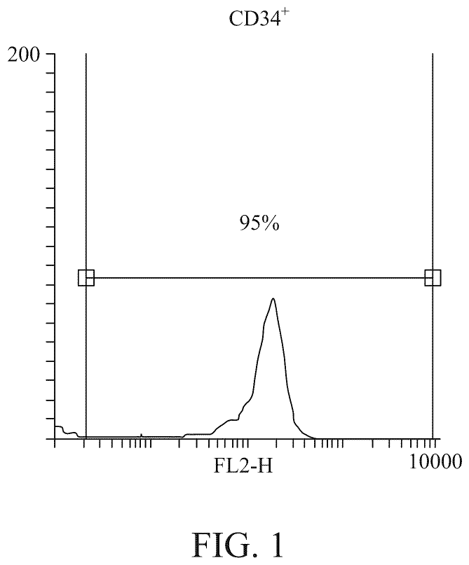

The collected cells were analyzed for CD34 expression by flow cytometry. The result is shown in FIG. 1.

According to FIG. 1, it is known that the expression of CD34 of cells collected through the aforementioned isolation method can be as high as 95%. Namely, through the aforementioned separation method, high purity hematopoietic stem cells can be surely separated from the donor's umbilical cord blood.

### Example 2

### Expansion of Hematopoietic Stem Cells

Respective hematopoietic stem cells were isolated from umbilical cord blood of different donors by the method of Example 1.

A commercially available serum-free medium for hematopoietic cell culture and expansion, StemSpan™ SFEM II, which is a modified Iscove's modified Dulbecco's medium (IMDM), was used as a basal medium. Human stem cell factor (SCF), human thrombopoietin (TPO), human Fms-like tyrosine kinase 3 ligand (Flt3L), human interleukin-3 (IL-3) and human interleukin-6 (IL-6) were added to the aforementioned basal medium in such a way that the respective final concentrations thereof were all 100 ng/ml to form a hematopoietic stem cell expansion medium.

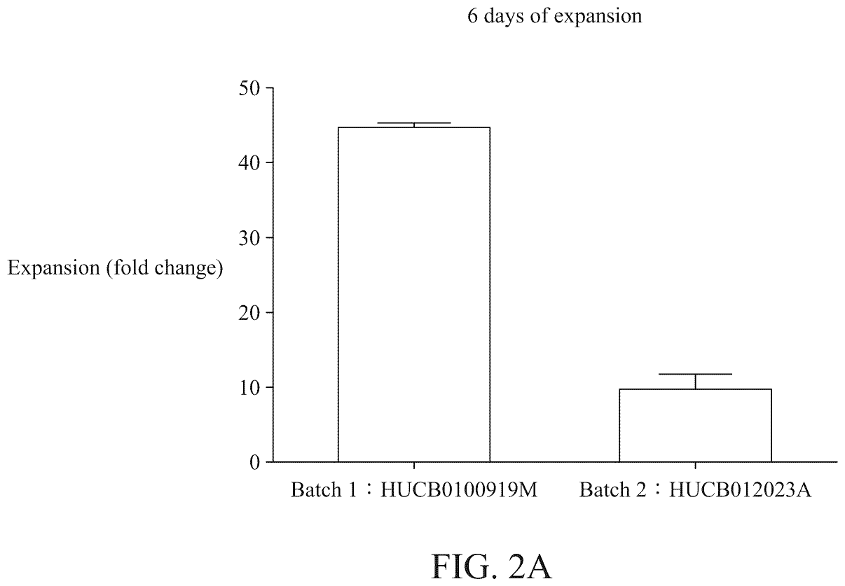

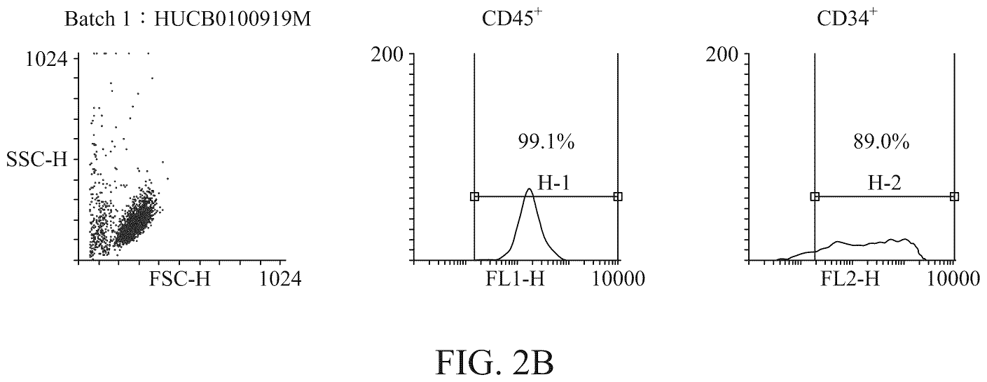

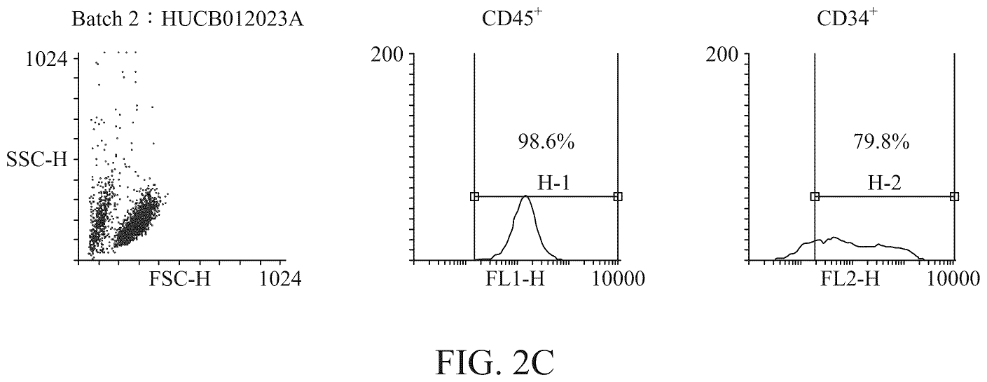

The hematopoietic stem cells isolated from umbilical cord blood of the donor with code HUCB0100919M and the donor with code HUCB012023A were respectively cultured with the hematopoietic stem cell expansion medium mentioned above for 6 days, and their expansion folds were calculated. The results are shown in FIG. 2A.

According to FIG. 2A, it is known that after the culture mentioned above, the expansion folds of the hematopoietic stem cells isolated from umbilical cord blood of the donor with code HUCB0100919M and the donor with code HUCB012023A respectively are 44±0.8 folds and 9.4±2.3 folds.

In addition, after the culture mentioned above, the hematopoietic stem cells isolated from umbilical cord blood of the donor with code HUCB0100919M and the donor with code HUCB012023A were respectively analyzed for CD45 and CD34 expressions of cells by flow cytometry. The results are respectively shown in FIG. 2B and FIG. 2C.

Based on FIG. 2B and FIG. 2C, it is known that, after the culture mentioned above, the expression levels of CD45 and CD34 of the hematopoietic stem cells isolated from the umbilical cord blood of both the donor with code HUCB0100919M and the donor with code HUCB012023A can be maintained at 98% or more and 79% or more than, respectively.

### Example 3

### Differentiation From Hematopoietic Stem Cells to γδ T Cells and Expansion of γδ T Cells

### 1. Formation of Coating in Culture Plate

Vascular cell adhesion molecule (VCAM), intercellular adhesion molecule (ICAM) and delta-like ligand 4 (DLL4) were added to phosphate buffered saline (PBS) to form a coating formation solution (final concentration: vascular cell adhesion molecule was 10 μg/mL; intercellular adhesion molecule was 10 μg/mL; delta-like ligand 4 was 0.1 μg/mL, 1 μg/mL or 10 μg/mL).

0.5 mL of the coating formation solution mentioned above was added to each well of a 24 well culture plate and stood at room temperature for 2 hours to form coating on the bottom surface of each well of the 24 well plate. After that, each well was washed once with phosphate buffered saline to wash away the substances not fixed on the bottom surface of each well, and the preparation of coating in the culture plate was completed.

### 2. Preparation of Differentiation-Promoting Medium

A commercially available serum-free medium for hematopoietic cell culture and expansion, StemSpan™ SFEM II, which is a modified Iscove's modified Dulbecco's medium (IMDM), was used as a basal medium. Stem cell factor (SCF), thrombopoietin, (TPO), Fms-like tyrosine kinase 3 ligand (Flt3L) and interleukin-7 (IL-7) were added to the aforementioned basal medium in such a way that the respective final concentrations thereof were all 8 ng/mL, were all 20 ng/mL, were all 50 ng/mL or are were 100 ng/mL to form a medium for promoting differentiation.

### 3. Differentiation Culture—Differentiation of Hematopoietic Stem Cells Into γδ T Cells

1×10⁴ hematopoietic stem cells obtained by the foregoing method were co-cultured with the foregoing differentiation-promoting medium in the 24 well culture plate with the foregoing coating (in the coating preparation solution, the concentrations of vascular cell adhesion molecule (VCAM), intercellular adhesion molecule (ICAM) and delta-like ligand 4 (DLL4) were all 10 μg/mL; content ratio of vascular cell adhesion molecule (VCAM), intercellular adhesion molecule (ICAM) and delta-like ligand 4 (DLL4) was 1:1:1) for 14 days. In the culture mentioned above, 0.5 mL of fresh differentiation-promoting medium mentioned above was added on the Day 4, and 0.5 mL of the cultured medium was replaced with fresh differentiation-promoting medium mentioned above (the final concentrations of stem cell factor, thrombopoietin, Fms-like tyrosine kinase 3 ligand and interleukin-7 were all 100 ng/mL) every 3 days.

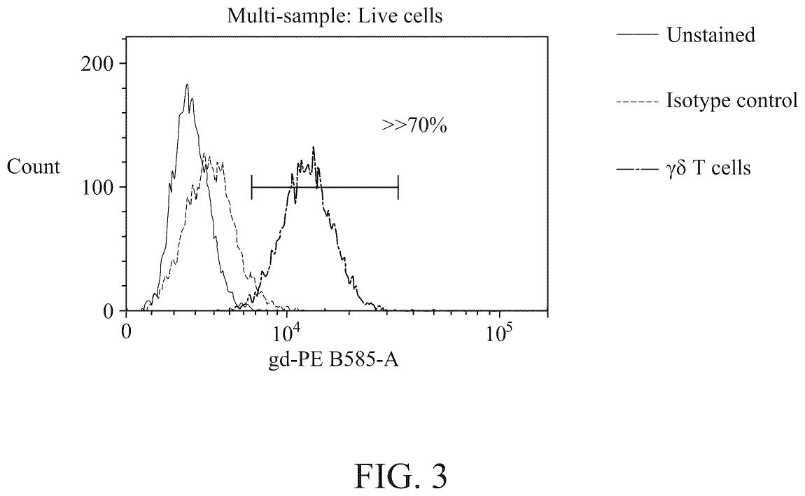

After the culture was completed, the obtained cells were analyzed for the expression of γδ TCR thereof by flow cytometry. The result is shown in FIG. 3.

Based on FIG. 3, it is known that after the culture mentioned above was completed, the expression level of γδ TCR is much higher than 70%, which means that the culture method mentioned above can indeed differentiate hematopoietic stem cells into γδ T cells.

Furthermore, in the circumstance of that the coating was replaced with coatings formed by coating formation solutions (the concentrations of both vascular cell adhesion molecule (VCAM) and intercellular adhesion molecule (ICAM) were maintained at 10 μg/mL) containing other concentrations of delta-like ligand 4 (DLL4) (0.1 μg/mL and 1 μg/mL) while other culture conditions were the same as above, the differentiation statuses of hematopoietic cells into γδ T cells were similar to the result mentioned above (not shown).

In addition, in the circumstances of that the final concentrations of stem cell factor, thrombopoietin, Fms-like tyrosine kinase 3 ligand and interleukin-7 in the differentiation-promoting medium mentioned above were all 8 ng/mL, were all 20 ng/ml and were all 50 ng/ml while other culture conditions were the same as above, the differentiation statuses of hematopoietic cells into γδ T cells were also similar to the result mentioned above (not shown).

### Example 4

### Evaluation of Toxic Effect of γδ T Cells Differentiated From Hematopoietic Stem Cells on Cancer Cells by Calcein AM Staining

In this experiment, lymphoma cells K562 or breast cancer cells MDA-MB-231 were used as target cells (labeled as T) for γδ T cells differentiated from hematopoietic stem cells.

After the target cells were stained with 1 M Calcein AM for 30 minutes, they were washed three times with PBS. After that, effector cells (labeled as E) (i.e., γδ T cells differentiated from hematopoietic stem cells) were co-cultured with target cells at an effector cell to target cell ratio (E:T ratio) of 2:1 or 5:1 to analyze the toxic ability of γδ T cells differentiated from hematopoietic stem cells on cancer cells.

After entering living cells, Calcein AM is capable of binding to intracellular calcium ions through hydrolyzation by esterases and thus emits fluorescence. When the effector cells generate a toxic effect on the target cells, that will cause the integrity of the target cell membrane to be damaged, and lead to the loss of calcium ions in the target cells, which will change the fluorescence intensity. Therefore, the change of fluorescence generated by Calcein AM can be used to evaluate the toxic ability of the effector cells on the target cells.

The intensity of fluorescence emission resulting from the rupture of target cells caused by 2% Triton-X 100 was defined as complete toxic killing (100%, value A) while the intensity of spontaneous fluorescence emission of target cells is defined as value B. Moreover, the intensity of fluorescence emission due to the toxicity of the effector cells is defined as the value C.

The toxic effect of the effector cells on the target cells was calculated by the following formula:

$$Toxic effect ( \% ) = ( C - B ) / ( A - B ) \times 100 .$$

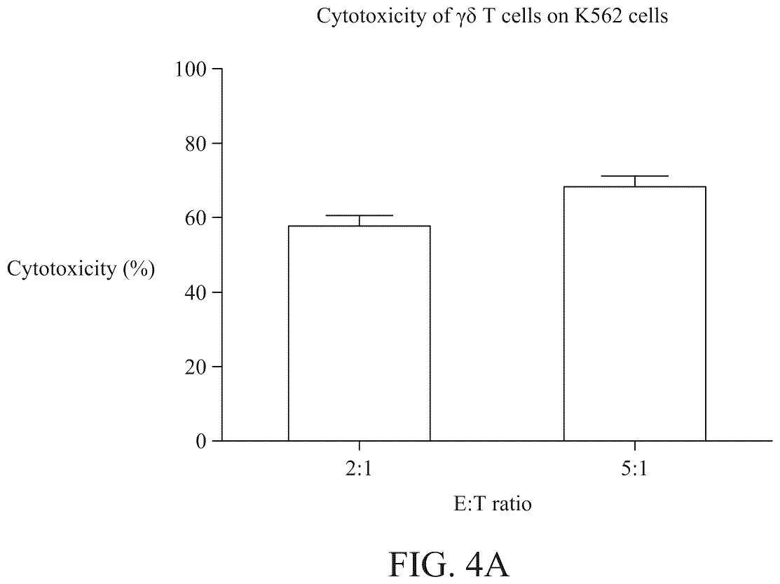

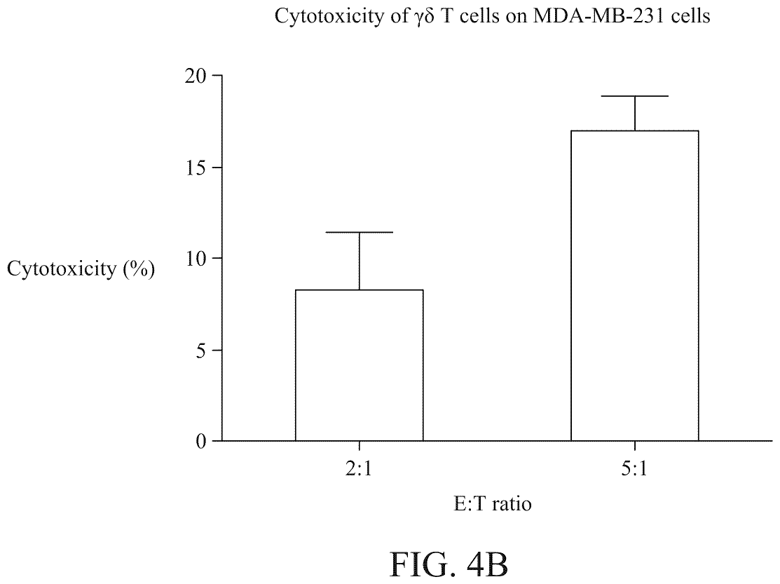

The toxic effects of γδ T cells differentiated from hematopoietic stem cells on lymphoma cells K562 and breast cancer cells MDA-MB-231 were evaluated by the aforementioned method. The results are shown in FIG. 4A and FIG. 4B, respectively.

According to FIG. 4A, it is known that the toxic effect of γδ T cells differentiated from hematopoietic stem cells on lymphoma cells K562 can reach 50% or more. Moreover, according to FIG. 4B, it is known that γδ T cells differentiated from hematopoietic stem cells have a dose-dependent toxic effect on breast cancer cells MDA-MB-231.

### Example 5

### Evaluation of Toxic Effect of γδ T CELLS DIFFERENTIATED FROM HEMATOPOIETIC Stem Cells on Cancer Cells by Real-Time Cell Analysis (RTCA)

In this experiment, breast cancer cells MDA-MB-231 were used as target cells (labeled as T) for γδ T cells differentiated from hematopoietic stem cells.

In the differentiation culture for the experimental group, hematopoietic stem cells isolated from the donor's umbilical cord blood were cultured with the culture plate containing a coating and the differentiation-promoting medium (hereinafter referred to as the medium of the present disclosure) used in “3. Differentiation culture-Differentiation of hematopoietic stem cells into γδ T cell” of Example 3 for a total of 13 days or 26 days to differentiate hematopoietic stem cells into γδ T cells. In the culture mentioned above, 0.5 mL of fresh differentiation-promoting medium mentioned above was added on the Day 4, and 0.5 mL of the cultured medium was replaced with fresh differentiation-promoting medium mentioned above every 3 days.

In the differentiation culture for the control group, except for using a commercially available serum-free medium for hematopoietic cell culture and expansion, Stem Span™ SFEM II, as the cell culture medium, other culture conditions were the same as those for the experimental group.

Real-time cell analysis is a technology based on impedance and microsensor electrodes, which can monitor statuses for cell adhesion, morphology and cell proliferation in a label-free, real-time and continuous manner. Cells are seeded into a special sensing well plate in the RTCA device, which can reflect the resistance changes caused by the growth and survival of the cells.

Effector cells (labeled as E) (i.e., γδ T cells differentiated from hematopoietic stem cells) were co-cultured with target cells at an effector cell to target cell ratio (E:T ratio) of 1:1 in the device for real-time cell analysis to analyze the toxic ability of γδ T cells differentiated from hematopoietic stem cells on cancer cells.

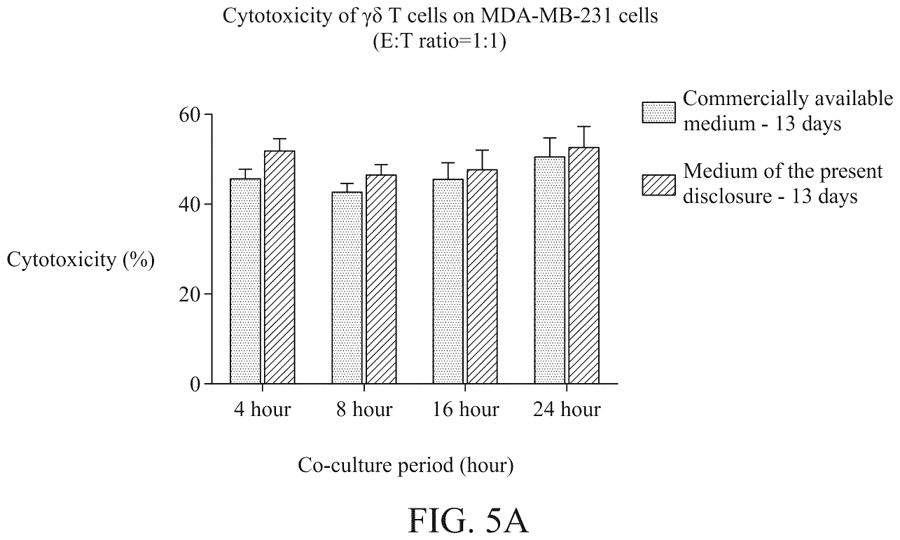

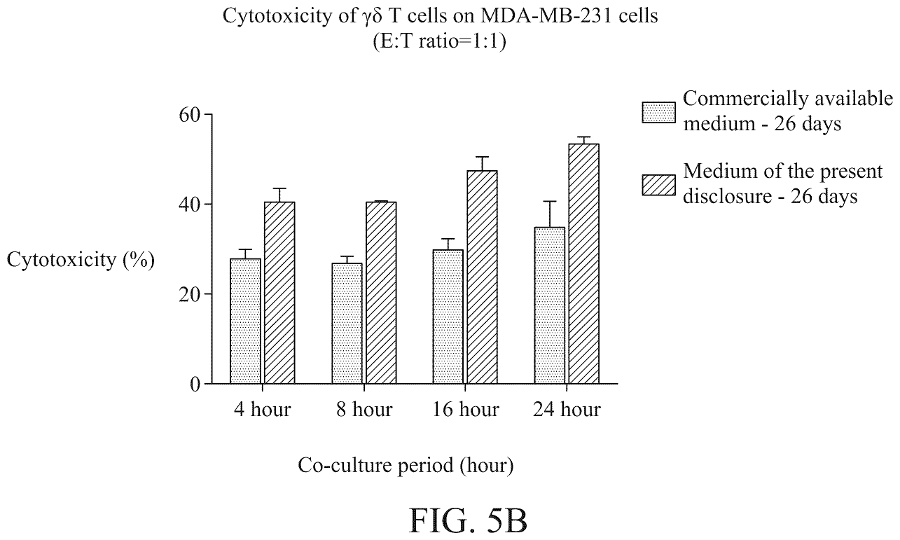

The toxic effect of γδ T cells differentiated from hematopoietic stem cells subjected to 13 days or 26 days of differentiation culture on breast cancer cells MDA-MB-231 was evaluated by the method mentioned above. The results are shown in FIG. 5A and FIG. 5B, respectively.

Based on FIG. 5A and FIG. 5B, it is known that compared to culturing by a commercially available medium, regardless of whether the differentiation culture is performed for 13 days or 26 days, the γδ T cells differentiated from the hematopoietic stem cells obtained by performing the differentiation culture with the medium of the present disclosure both have better cancer cell toxic ability.

### Example 6

### Effect of Intercellular Adhesion Molecule (ICAM) in Coatings in Differentiation Culture on the Efficiency of Differentiation of Hematopoietic Stem Cells Into γδ T Cells

In this experiment, the differentiation culture for the experimental group and the control group was basically the same as the culture manner in “3. Differentiation culture—Differentiation of hematopoietic stem cells into γδ T cell” of Example 3, with the only difference being the medium and coatings used.

Specifically, the medium prepared in “2. Preparation of differentiation-promoting medium” in Example 3 (herein referred to as the medium of the present disclosure, containing 100 ng/mL stem cell factor (SCF); 100 ng/mL thrombopoietin (TPO); 100 ng/ml Fms-like tyrosine kinase 3 ligand (Flt3L) and 100 ng/mL interleukin-7 (IL-7)) was used in the experimental group, and the experimental group was further divided into three sub groups, which were no coating group, VDI group and VD group, respectively. For the no coating, no coating was used during the culture. For the VDI group, a coating containing vascular cell adhesion molecule (VCAM), delta-like ligand 4 (DLL4) and intercellular adhesion molecule (ICAM) (in the coating preparation solution, the concentrations of vascular cell adhesion molecule (VCAM), delta-like ligand 4 (DLL4) and intercellular adhesion molecule (ICAM) were all 10 μg/mL; the content ratio of vascular cell adhesion molecule (VCAM), delta-like ligand 4 (DLL4) and intercellular adhesion molecule (ICAM)) was 1:1:1) was used during the culture while for the VD group, a coating only containing vascular cell adhesion molecule (VCAM) and delta-like ligand 4 (DLL4) (in the coating preparation solution, the concentrations of vascular cell adhesion molecule (VCAM) and delta-like ligand 4 (DLL4) were both 10 μg/mL; the content ratio of vascular cell adhesion molecule (VCAM) to delta-like ligand 4 (DLL4) was 1:1) was used during the culture.

By contrast, for the control group, a commercially available serum-free medium for hematopoietic cell culture and expansion, StemSpan™ SFEM II (herein referred to as the commercially available medium), was used, and the control group was also further divided into three sub groups, which also were no coating group, VDI group and VD group, respectively. For the no coating, no coating was used during the culture. For the VDI group, a coating containing vascular cell adhesion molecule (VCAM), delta-like ligand 4 (DLL4) and intercellular adhesion molecule (ICAM) (in the coating preparation solution, the concentrations of vascular cell adhesion molecule (VCAM), delta-like ligand 4 (DLL4) and intercellular adhesion molecule (ICAM) were all 10 μg/mL; the content ratio of vascular cell adhesion molecule (VCAM), delta-like ligand 4 (DLL4) and intercellular adhesion molecule (ICAM)) was 1:1:1) was used during the culture while for the VD group, a coating only containing vascular cell adhesion molecule (VCAM) and delta-like ligand 4 (DLL4) (in the coating preparation solution, the concentrations of vascular cell adhesion molecule (VCAM), delta-like ligand 4 (DLL4) were both 10 μg/mL; the content ratio of vascular cell adhesion molecule (VCAM) to delta-like ligand 4 (DLL4) was 1:1) was used during the culture.

The preparation manners for the different coatings were basically the same as the preparation method recited in “1. Formation of coating in culture plate” of Example 3 with the only difference being whether or not to add intercellular adhesion molecule (ICAM).

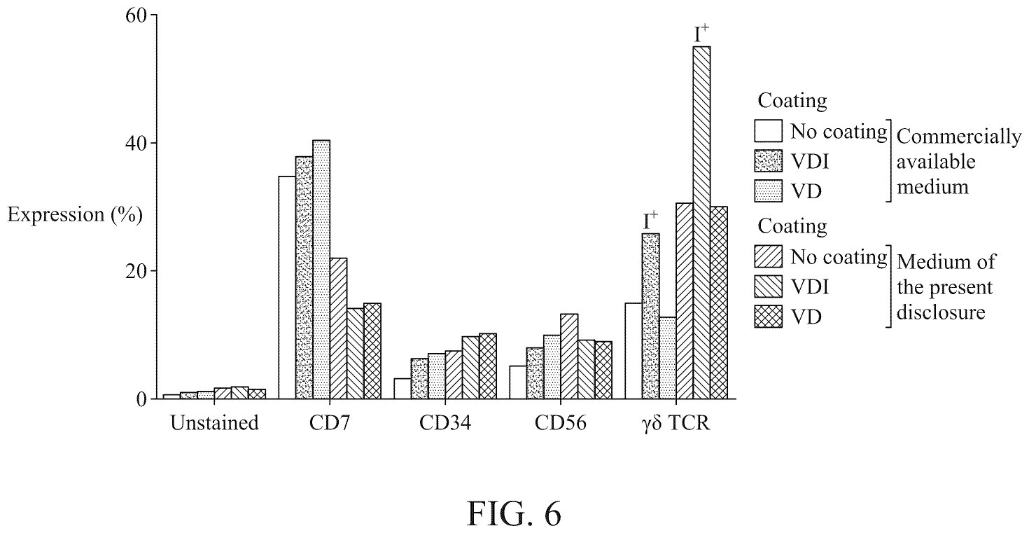

After the differentiation culture was completed, the cells in each group were analyzed by flow cytometry for their unstained status (negative control group) and expressions of CD7, CD34, CD56 and γδ TCR. The results are shown in FIG. 6.

According to FIG. 6, it is known that compared to culturing under a coating containing only vascular cell adhesion molecule (VCAM) and delta-like ligand 4 (DLL), hematopoietic stem cells which cultured under a coating containing vascular cell adhesion molecule (VCAM), delta-like ligand 4 (DLL4) and intercellular adhesion molecule (ICAM) have higher expression of γδ TCR and show lower level of CD7, that suggests that intercellular adhesion molecule (ICAM) is capable of allowing hematopoietic stem cells to differentiate from pre-mature T cells into more mature γδ T cells.

In addition, based on FIG. 6, it is also known that compared to culturing with commercially available medium, culturing hematopoietic stem cells with the medium of the present disclosure can have better differentiation efficiency into γδ T cells.

### Example 7

### Effect of Delta-Like Ligand 4 (DLL4) in Coatings in Differentiation Culture on the Efficiency of Differentiation of Hematopoietic Stem Cells Into γδ T Cells

In this experiment, the differentiation culture was basically the same as the culture manner in “3. Differentiation culture-Differentiation of hematopoietic stem cells into γδ T cell” of Example 3, with the only difference being the coatings used.

Specifically, there were two experimental groups in this experiment, IV group and IVD group. The medium prepared in “2. Preparation of differentiation-promoting medium” in Example 3 (herein referred to as the medium of the present disclosure, containing 100 ng/mL stem cell factor (SCF); 100 ng/mL thrombopoietin (TPO); 100 ng/mL Fms-like tyrosine kinase 3 ligand (Flt3L) and 100 ng/mL interleukin-7 (IL-7)) was used in the two experimental group.

However, for the IV group, a coating only containing intercellular adhesion molecule (ICAM) and vascular cell adhesion molecule (VCAM) (in the coating preparation solution, the concentrations of intercellular adhesion molecule (ICAM) and vascular cell adhesion molecule (VCAM) were both 10 μg/mL; the content ratio of intercellular adhesion molecule (ICAM) to vascular cell adhesion molecule (VCAM) was 1:1) was used during the culture while for the IVD group, a coating containing intercellular adhesion molecule (ICAM), vascular cell adhesion molecule (VCAM), and delta-like ligand 4 (DLL4) (in the coating preparation solution, the concentrations of intercellular adhesion molecule (ICAM), vascular cell adhesion molecule (VCAM) and delta-like ligand 4 (DLL4) were all 10 μg/mL; the content ratio of intercellular adhesion molecule (ICAM), vascular cell adhesion molecule (VCAM) and delta-like ligand 4 (DLL4) was 1:1:1) was used during the culture.

The preparation manners for the different coatings were basically the same as the preparation method recited in “1. Formation of coating in culture plate” of Example 3 with the only difference being whether or not to add delta-like ligand 4 (DLL4).

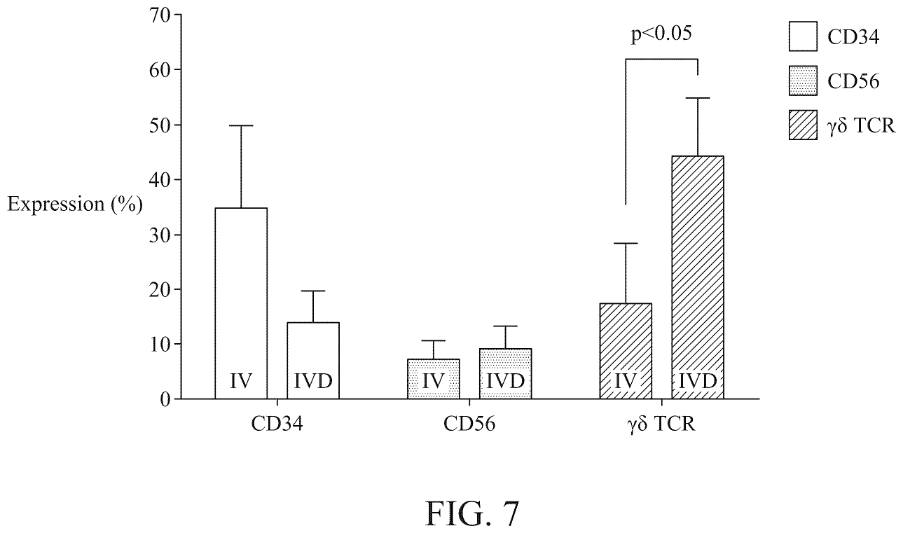

After the differentiation culture was completed, the cells in each group were analyzed by flow cytometry for their expressions of CD34, CD56 and γδ TCR. The results are shown in FIG. 7.

Based on FIG. 7, it is known that compared to culturing under a coating containing only intercellular adhesion molecule (ICAM) and vascular cell adhesion molecule (VCAM), hematopoietic stem cells which cultured under a coating containing intercellular adhesion molecule (ICAM), vascular cell adhesion molecule (VCAM) and delta-like ligand 4 (DLL4) have higher expression of γδ TCR and show lower level of CD7, that suggests that delta-like ligand 4 (DLL4) is capable of allowing hematopoietic stem cells to differentiate from pre-mature T cells into more mature γδ T cells.

It will be apparent to those skilled in the art that various modifications and variations can be made to the disclosed embodiments. It is intended that the specification and examples be considered as exemplary only, with the true scope of the disclosure being indicated by the following claims and their equivalents.

What is claimed is:

1. A method for preparing an immune cell differentiated from a stem cell, comprising:
(a) forming a coating containing a matrix on a surface; and
(b) culturing a stem cell on the coating in the presence of a first cytokine combination to differentiate the stem cell into an immune cell, wherein the immune cell comprises a γδ T cell (gamma delta T cell, GDT cell),
wherein the matrix comprises:
vascular cell adhesion molecule (VCAM);
intercellular adhesion molecule (ICAM); and
delta-like ligand 4 (DLL4);
wherein the first cytokine combination comprises:
stem cell factor (SCF);
thrombopoietin (TPO);
Fms-like tyrosine kinase 3 ligand (Flt3L); and
interleukin-7 (IL-7); and
wherein a source of the stem cell comprises umbilical cord blood or an induced pluripotent stem cell (iPSC), and the stem cell is CD34 positive.

2. The method for preparing an immune cell differentiated from a stem cell as claimed in claim 1, wherein the coating is formed by a coating formation solution, and the coating formation solution contains the matrix.

3. The method for preparing an immune cell differentiated from a stem cell as claimed in claim 1, wherein in the coating formation solution, the concentration of the vascular cell adhesion molecule is 0.05-50 μg/mL, the concentration of the intercellular adhesion molecule is 0.05-50 μg/mL, and the concentration of the delta-like ligand 4 is 0.05-50 μg/mL.

4. The method for preparing an immune cell differentiated from a stem cell as claimed in claim 1, wherein in step (b), the concentration of the stem cell factor is 1-500 ng/mL, the concentration of the thrombopoietin is 1-500 ng/mL, the concentration of the Fms-like tyrosine kinase 3 ligand is 1-500 ng/mL, and the concentration of the interleukin-7 is 1-500 ng/mL.

5. The method for preparing an immune cell differentiated from a stem cell as claimed in claim 1, wherein in step (b), the stem cell is cultured at 35-37° C. for 7-42 days.

6. The method for preparing an immune cell differentiated from a stem cell as claimed in claim 1, further comprising (b′) screening the stem cell from the source of the stem cell before step (b).

7. The method for preparing an immune cell differentiated from a stem cell as claimed in claim 1, further comprising (b″) expanding the stem cell before step (b).

8. The method for preparing an immune cell differentiated from a stem cell as claimed in claim 1, before step (b), further comprising:
(b′) screening the stem cell from the source of the stem cell; and
(b″) expanding the stem cell after step (b′).

9. The method for preparing an immune cell differentiated from a stem cell as claimed in claim 7, wherein a procedure for expanding the stem cell comprises:
(b″-1) culturing the stem cell in the presence of a second cytokine combination.

10. The method for preparing an immune cell differentiated from a stem cell as claimed in claim 9, wherein the second cytokine combination comprises:
stem cell factor;
thrombopoietin;
Fms-like tyrosine kinase 3 ligand;
interleukin-3 (IL-3); and
interleukin-6 (IL-6).

11. The method for preparing an immune cell differentiated from a stem cell as claimed in claim 10, wherein in step (b″), the concentration of the stem cell factor is 1-500 ng/mL, the concentration of the thrombopoietin is 1-500 ng/mL, the concentration of the Fms-like tyrosine kinase 3 ligand is 1-500 ng/mL, the concentration of the interleukin-3 is 1-500 ng/mL, and the concentration of the interleukin-6 is 1-500 ng/mL.

12. The method for preparing an immune cell differentiated from a stem cell as claimed in claim 9, wherein in step (b″-1), the stem cell is cultured at 35-37° C. for 7-42 days.

13. A kit for preparing an immune cell differentiated from a stem cell, comprising:
a coating formation sub-kit for forming a coating on a surface; and
a stem cell differentiation sub-kit,
wherein the coating formation sub-kit comprises: a matrix component for forming a coating formation solution with a solvent,
wherein the matrix component comprises:
vascular cell adhesion molecule (VCAM);
intercellular adhesion molecule (ICAM); and
delta-like ligand 4 (DLL4); and
wherein the coating formation solution is used to coat the surface to form the coating on the surface, and
wherein the stem cell differentiation sub-kit comprises: a first cytokine combination for adding to a medium to form a stem cell differentiation medium,
wherein the first cytokine combination comprises:
stem cell factor;
thrombopoietin;
Fms-like tyrosine kinase 3 ligand; and
interleukin-7.

14. The kit for preparing an immune cell differentiated from a stem cell as claimed in claim 13, wherein in the coating formation solution, the concentration of the vascular cell adhesion molecule is 0.05-50 μg/mL, the concentration of the intercellular adhesion molecule is 0.05-50 μg/mL, and the concentration of the delta-like ligand 4 is 0.05-50 μg/mL.

15. The kit for preparing an immune cell differentiated from a stem cell as claimed in claim 13, wherein in the stem cell differentiation medium, the concentration of the stem cell factor is 1-500 ng/mL, the concentration of the thrombopoietin is 1-500 ng/mL, the concentration of the Fms-like tyrosine kinase 3 ligand is 1-500 ng/ml, and the concentration of the interleukin-7 is 1-500 ng/mL.

16. The kit for preparing an immune cell differentiated from a stem cell as claimed in claim 13, wherein the coating formation sub-kit further comprises the solvent to form the coating formation solution with the matrix component.

17. The kit for preparing an immune cell differentiated from a stem cell as claimed in claim 13, wherein the stem cell differentiation sub-kit further comprises the medium.

18. The kit for preparing an immune cell differentiated from a stem cell as claimed in claim 13, further comprising a stem cell expansion sub-kit,
wherein the stem cell expansion sub-kit comprises: a second cytokine combination for adding to another medium to form a stem cell expansion medium,
wherein the second cytokine combination comprises:
stem cell factor;
thrombopoietin;
Fms-like tyrosine kinase 3 ligand;
interleukin-3; and
interleukin-6.

19. The kit for preparing an immune cell differentiated from a stem cell as claimed in claim 18, wherein in the stem cell expansion medium, the concentration of the stem cell factor is 1-500 ng/mL, the concentration of the thrombopoietin is 1-500 ng/mL, the concentration of the Fms-like tyrosine kinase 3 ligand is 1-500 ng/ml, the concentration of the interleukin-3 is 1-500 ng/ml, and the concentration of the interleukin-6 is 1-500 ng/mL.

20. The kit for preparing an immune cell differentiated from a stem cell as claimed in claim 18, wherein the stem cell expansion sub-kit further comprises the other medium.
